(19) United States
(12) Patent Application Publication
(10) Pub. No.: US 2025/0332291 A1
(43) Pub. Date: Oct. 30, 2025

(54) MONOCLONAL ANTIBODIES FOR TARGETING THE CARDIAC CONDUCTION SYSTEM

(71) Applicant: THE BOARD OF TRUSTEES OF THE LELAND STANFORD JUNIOR UNIVERSITY, Stanford, CA (US)

(72) Inventors: Sean M. WU, Stanford, CA (US); William R. GOODYER, Stanford, CA (US)

(21) Appl. No.: 18/847,892

(22) PCT Filed: Mar. 21, 2023
(86) PCT No.: PCT/US2023/015747

§ 371 Date: Sep. 17, 2024

Related U.S. Application Data
(60) Provisional application No. 63/322,297, filed on Mar. 22, 2022.

(51) Int. Cl. A61K 49/00; C07K 16/28

(52) U.S. Cl. CPC A61K 49/0058; C07K 16/2803; C07K 16/286; C07K 2317/55

(57) ABSTRACT

Antibodies specific to human contactin2 (CNTN2), and imaging and/or diagnostic reagents and compositions visualizing the CCS cells, therapeutic products and compositions comprising one or more of the antibodies. Methods for delivering therapeutic agents to the CCS cells. The disclosure further provides methods for visualizing the CCS cells in vivo in real time, including in a subject undergoing a cardiothoracic surgery or other cardiac intervention.

### CROSS-REFERENCE TO RELATED APPLICATIONS

This application claims the benefit of priority from U.S. Provisional Patent Application 63/322,297 filed Mar. 22, 2022, the entire disclosure of which is herein incorporated by reference in its entirety.

### STATEMENT OF GOVERNMENT INTEREST

This invention was made with Government support under contract HL153785 awarded by the National Institutes of Health. The Government has certain rights in the invention.

### INCORPORATION OF .XML SEQUENCE LISTING

This application contains a Sequence Listing which has been submitted electronically in .XML format. Said .XML sequence listing, created on Mar. 9, 2023, is named “4227.148487PCT.xml” and is 34,771 bytes in size. The sequence listing contained in this .XML file is part of the specification and is hereby incorporated by reference herein in its entirety.

### TECHNICAL FIELD

The invention relates to monoclonal antibodies for targeting the cardiac conduction system (CCS) cells, imaging and/or therapeutic reagents comprising the monoclonal antibodies, and compositions and methods for delivering therapeutic drugs and/or detection agents to the CCS cells. The invention further relates to compositions and methods visualizing the CCS cells in vivo in real time, including in a subject undergoing a cardiothoracic surgery or other cardiac intervention.

### BACKGROUND

The cardiac conduction system (CCS) is made up of specialized heart cells that establish the rhythmic beating of the heart through coordinated contraction of its chambers. The cardiac conduction system (CCS) is comprised of distinct components including the sinoatrial node (SAN), atrioventricular node (AVN), His bundle (HIS), bundle branches (BB) and Purkinje fibers (PF). The CCS is essential for the formation and normal function of the heart and disturbance to the CCS can result in severe clinical manifestations including arrhythmias, decreased cardiac output and even sudden death. Despite an essential role for the CCS in heart development and function, the CCS has remained difficult to study due to inherent obstacles including small cell numbers, large cell type heterogeneity, complex anatomy and difficulty in isolation. Each component of the CCS consists of unique cardiac cell types with their own physiologic and electrochemical properties.

The CCS is made up of specialized heart cells that establish the rhythmic beating of the heart through coordinated contraction of its chambers.¹ Damage to the CCS can result in decreased cardiac function, life-long need for electronic pacemakers as well as fatal arrhythmias.² The CCS is invisible to the naked eye and, as such, is often accidentally damaged during invasive cardiac procedures, including surgeries and transcatheter-based interventions, for which there are estimated to be over 900,000 yearly in the US alone. In fact, postoperative heart block, secondary to accidental surgical damage of the atrioventricular node (AVN), complicates roughly 1-3% of all congenital heart disease (CHD) surgeries and 4-24% of more complex repairs.^{3,4} Similarly, many adult cardiac interventions including transcatheter valve replacements and septal ablations are plagued with a similarly high risk of CCS damage (˜8-25%). 5 7 Use of advanced imaging, including cardiac magnetic resonance imaging (MRI) and computerized tomography (CT), have become increasingly important in preoperative planning for cardiac interventions, however these imaging modalities, despite their exquisite resolution, are still unable to detect the CCS, leaving the interventionalists effectively ‘blind’ as to its location within the heart. Current standard of care remains the use of anatomical landmarks to approximate the location of the CCS. To date, there exists no commercially available method for cardiac proceduralists to visualize the CCS in the operating suite nor during catheter-based interventions.

Optical imaging using molecularly targeted antibodies conjugated to fluorescent dyes is a burgeoning technology within translational medicine, providing potential opportunities in diagnostics (e.g. tumor burden detection) and image-guided surgery in order to improve surgical outcomes.⁸ The vast majority of research into optical imaging to date has focused on real-time tumor detection for intraoperative image-guided oncologic resections, with several of these agents currently being evaluated in clinical trials.⁹ Optical imaging agents theoretically also have incredible potential for visualizing and thus sparing normal tissues often inadvertently damaged during invasive procedures. Particular hurdles to broadening this technology to more complex structures such as the heart include: i). the lack of distinguishing surface markers; ii). cell-type heterogeneity; iii). the depth below the tissue surface limiting both the delivery and visualization of the fluorescent signal; and iv). a complex three-dimensional anatomy.^{10,11}

### SUMMARY

This disclosure addresses at least some of the unmet needs in the art.

In one aspect, this disclosure provides an antibody or an antigen-binding fragment thereof, having a specific binding activity to human contactin 2 (CNTN2) protein with the amino acid sequence of SEQ ID NO: 9, the antibody or the antigen-binding fragment thereof comprising:
a). at least a first polypeptide comprising a light chain variable region (V_{L}); and
b). at least a second polypeptide comprising a heavy chain variable region (V_{H}),
wherein the first polypeptide is linked to the second polypeptide,
wherein the light chain variable region (V_{L}) of the first polypeptide comprises at least one from the following four amino acid sequences:
   the amino acid sequence with SEQ ID NO: 1 or a variant thereof with at least 70% amino acid sequence identity to SEQ ID NO: 1;
   the amino acid sequence with SEQ ID NO: 5 or a variant thereof with at least 70% amino acid sequence identity to SEQ ID NO: 5;
   an amino acid sequence comprising Complementarity-Determining Region 1 (CDR1) with SEQ ID NO: 10 or a variant thereof with at least 90% amino acid sequence identity to SEQ ID NO: 10, Complementarity-Determining Region 2 (CDR2) with SEQ ID NO: 11 or a variant thereof with at least 90% amino acid sequence identity to SEQ ID NO: 11, and Complementarity-Determining Region 3 (CDR3) with SEQ ID NO: 13 or a variant thereof with at least 90% amino acid sequence identity to SEQ ID NO: 13; or
   an amino acid sequence comprising CDR1 with SEQ ID NO: 15 or a variant thereof with at least 90% sequence identity to SEQ ID NO: 15, CDR2 with SEQ ID NO: 16 or a variant thereof with at least 90% sequence identity to SEQ ID NO: 16, and CDR3 with SEQ ID NO: 17 or a variant thereof with at least 90% sequence identity to SEQ ID NO: 17; and
wherein the heavy chain variable region (V_{H}) of the second polypeptide comprises at least one from the following four amino acid sequences:
   an amino acid sequence with SEQ ID NO: 3 or a variant thereof with at least 70% amino acid sequence identity to SEQ ID NO: 3;
   an amino acid sequence with SEQ ID NO: 7 or a variant thereof with at least 70% amino acid sequence identity to SEQ ID NO: 7;
   an amino acid sequence comprising CDR1 with SEQ ID NO: 13 or a variant thereof having at least 90% sequence identity to SEQ ID NO: 13, and CDR2 with SEQ ID NO: 14 or a variant thereof with at least 90% identity to SEQ ID NO: 14; or
   an amino acid sequence comprising CDR1 with SEQ ID NO: 18 or a variant thereof with at least 90% sequence identity to SEQ ID NO: 18, and CDR2 with SEQ ID NO: 19 or a variant thereof with at least 90% sequence identity to SEQ ID NO: 19.

Some preferred embodiments of the antibody or the antigen-binding fragment thereof include those wherein the first polypeptide is linked to the second polypeptide by a disulfide bond or by a peptide linker and/or wherein the antibody or the antigen-binding fragment further comprises a third polypeptide comprising the light chain variable region (V_{L}), the third polypeptide being linked to a fourth polypeptide, the fourth polypeptide comprising the heavy variable region (V_{H}) and wherein the second polypeptide and the fourth polypeptide are linked.

Some preferred embodiments of the antibody or the antigen-binding fragment include those, wherein the first polypeptide further comprises at least a portion of a light chain constant region (C_{L}) and wherein the second polypeptide further comprises a least a portion of a heavy chain constant region (C_{H1}).

In some embodiments, the antibody or the antigen-binding fragment thereof may comprise two polypeptides a). and two polypeptides b)., and wherein the two polypeptides b). are linked.

Preferably, the antibody or the antigen-binding fragment thereof may be a human monoclonal antibody. A particularly preferred embodiment of the antibody or the antigen-binding fragment thereof includes a human monoclonal fragment antigen-binding region (Fab) composed of the first polypeptide linked to the second polypeptide, wherein the first polypeptide further comprises at least a portion of a light chain constant region (C_{L}) and wherein the second polypeptide further comprises a least a portion of a heavy chain constant region (C_{H1}).

In some particularly preferred embodiments, the antibody or the antigen-binding fragment thereof may comprise the first polypeptide comprising the amino acid sequence with SEQ ID NO: 1 or 5, or a variant amino acid sequence with at least 90% or at least 95% amino acid sequence identity to SEQ ID NO: 1 or 5; and the second polypeptide comprising the amino acid sequence with SEQ ID NO: 3 or 7, or a variant amino acid sequence with at least 90% or at least 95% amino acid sequence identity to SEQ ID NO: 3 or 7.

Examples of suitable antigen-binding antibody fragments include, but are not limited to, Fab, F(ab)₂, scFv, a diabody and/or any other antibody fragments having a specific binding activity to human contactin 2 (CNTN2) protein with the amino acid sequence of SEQ ID NO: 9. In some embodiments, the antibody is a polyclonal antibody, monoclonal antibody, a single-chain antibody, a chimeric antibody, or a humanized monoclonal antibody. Preferably, the antibody is recombinant, isolated and/or purified human monoclonal antibody or human monoclonal Fab.

In another aspect, this disclosure relates to a reagent useful for therapeutic, imaging and/or diagnostic applications, the reagent comprising the antibody or the antigen binding fragment thereof according to this disclosure, wherein the antibody or the antigen binding fragment thereof is conjugated with one or more of a detection agent and/or a therapeutic drug. In some preferred embodiments, the antibody or the antigen binding fragment therefore may be conjugated with a near-infrared dye (NIR), a superparamagnetic iron oxide nanoparticle (SPION), a gold nanoparticle, saporin, an antiarrhythmic drug, a ribonucleic acid (RNA), deoxyribonucleic acid (DNA) or CAS9 enzyme-single guide complex. The antibody or the antigen binding fragment thereof may be conjugated directly or indirectly for example by being displayed at the surface of a carrier enclosing the therapeutic drug or the detection agent, preferably a liposome carrier. The reagents of this disclosure may include those, wherein the antibody is conjugated directly to the detection agent or the therapeutic drug. The reagents of this disclosure also include those, wherein the antibody is conjugated to the detection agent and/or therapeutic drug indirectly via a linker or another molecule such as for example as biotin or streptavidin.

In particularly preferred embodiments of the reagent, the antibody or the antigen binding fragment thereof may be conjugated directly or indirectly to amiodarone, procainamide or near-infrared dye (NIR).

Further suitable examples of a detection agent that may be used in the reagent or a composition according to this disclosure include those, wherein the detection agent is one or more of the following: an imaging dye comprising a chromophore, a fluorophore, a tag, a radioactive isotope, a small molecule, a biomolecule, and/or a nanoparticle. Preferably, the imaging and/or diagnostic reagents include those, wherein the detection agent comprises a biocompatible near-infrared fluorophore.

Further suitable examples of a therapeutic drug that may be used in the reagent or a composition according to this disclosure include those, wherein the therapeutic drug is a small molecule or a biomolecule, digoxin, a calcium channel blocker, a beta blocker, an anti-arrhythmic drug, or RNA or DNA that can silence or activate at least one biologic function of the CCS cell. The therapeutic drug may be an antiarrhythmics drug, an CCS agonist drug, and/or an anti-inflammatory drug, diltiazem, verapamil, metoprolol, carvedilol, atenolol, digoxin, adenosine, dipyridamole, diphtheria toxin A, methotrexate, doxorubicin, isoproterenol, epinephrine, glucocorticoid, cyclosporin A, tacrolimus or saporin.

In yet another aspect, this disclosure relates to a composition comprising one or more antibodies or the antigen-binding fragment thereof according to this disclosure, or one or more reagents according to this disclosure, and one or more excipients.

Some compositions may comprise from 0.1 wt % to 99.9 wt % of the antibody or the antigen-binding fragment thereof or the regent, and from 0.1 wt % to 99.9 wt % of the one or more excipients. The compositions may include those which are formulated for oral, topical, local or systemic delivery to a subject. In some embodiments, the compositions may comprise one or more of the following excipients: water, a buffer, a solvent, a carrier, a bulking agent and/or a filler.

The compositions and the reagents according to this disclosure may be useful for treating subjects in need of treatment for the CCS related disorder or disease, including cardiac arrhythmia, accelerated heart rhythm, heart block, or atrial or ventricular fibrillation.

The compositions and the reagents according to this disclosure may be also useful for visualizing the CCS in a subject, including subjects that are undergoing a cardiothoracic surgery or a catheter procedure, or in preparation for a cardiothoracic surgery or procedure, e.g., the CCS mapping as may be helpful in cardiac ablation procedures.

In yet another aspect, this disclosure provides a method of treating a subject, the method comprising administering to the subject one or more compositions according to this disclosure. In some embodiments, the composition may be administered orally, topically, locally or systemically. In some embodiments, the composition may be administered in an amount from about 0.05 mg to about 100 mg of the detection agent and/or the therapeutic drug per one kilogram of the subject body weight.

In some preferred embodiments, the methods of treatment may include treating a patient in need for treatment of the CCS related disorder or disease, the method comprising administering to the patient one or more compositions according to this disclosure. The methods include those, wherein the patient is treated for one of the following diseases: cardiac arrhythmia, accelerated heart rhythm, heart block, or atrial or ventricular fibrillation. The methods include those, wherein the patient is administered from 0.05 mg to about 100 mg of the therapeutic drug per one kilogram of the body weight.

Preferred methods also include methods for visualizing the CCS in a subject, the method comprising administering to the subject one or more of the following: the reagent according to this disclosure and/or the composition according to this disclosure and comprising the reagent. The methods may further comprise detecting the CCS in the subject in real time.

The methods may include those, wherein the detecting comprises one of more of the following: ultrasound, computed tomography, illuminating with a scope the CCS of the subject under UV, visible, and/or infrared light; and/or directly shining the UV, visible, and/or infrared light at the CCS of the subject. The methods include those, wherein the method may further comprise capturing images of the CCS in real time with camera. The methods may include those, wherein the subject is undergoing a cardiothoracic surgery or a catheter procedure, including cardiac ablation. The methods include those, wherein the subject is administered from 0.05 mg to about 100 mg of the reagent per one kilogram of the body weight.

In yet another aspect, the present disclosure provides a method for preparing the reagent according to this disclosure, the method may comprise conjugating directly or indirectly the antibody or the antigen binding fragment thereof to a detection agent or a therapeutic drug. In some embodiments, the detection agent or the therapeutic drug may be conjugated in a weight-by-weight ratio in the range from about 1:1000 to about 1000:1 of the detection agent or the therapeutic drug to the antibody or the antigen binding fragment thereof. In some embodiments, the antibody or the antigen-binding fragment thereof may be first biotinylated and then conjugated via biotin/streptavidin coupling with the therapeutic drug or the detection agent which has been coupled with streptavidin.

In yet another aspect, this disclosure relates to a recombinant nucleic acid comprising the nucleic acid with SEQ ID NO. 2, SEQ ID NO. 4, SEQ ID NO. 6 and/or SEQ ID NO. 8, or a nucleic acid variant with at least 70%, preferably at least 80%, more preferably at least 90% and most preferably at least 95% nucleic acid sequence identity to SEQ ID NO. 2, SEQ ID NO. 4, SEQ ID NO. 6 and/or SEQ ID NO. 8. The nucleic acid may be incorporated into a nucleic acid construction, such as for example as a recombinant plasmid. In the construct, the nucleic acid may be operably linked to a promoter suitable for controlling expression of the nucleic acid.

In yet another aspect, the present disclosure relates to a recombinant cell comprising at least one nucleic acid or nucleic acid construction according to this disclosure. Examples of cells include, but are not limited to, bacterial cells, yeast cells, and mammalian cells. In some embodiments, the nucleic acid may be integrated into the cell genome.

In yet another aspect, this disclosure relates to a method for visualizing the cardiac conduction system (CCS) in a subject, the method comprising:
administering to the subject the composition according to this disclosure, the composition comprising at least one contactin 2 specific antibody or its antigen-binding fragment, or administering to the subject a composition comprising one or more antibodies with a specific binding activity to human neurotrimin protein (NTM) or human neuroplastin protein (NPTN); and
visualizing the CCS in the subject in real time by contacting the subject with ultrasound, computed tomography, visible, UV and/or infrared light.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Systemic Injection of mCntn2-800 in Mice Labels the CCS In Vivo. (A) Antibody-dye conjugate (mCntn2-800) consists of a near-infrared (NIR) dye to an antibody against the CCS-specific surface marker Cntn2 (Contactin 2). (B) Experimental work flow. (C) Whole-body biodistribution of other tissue types, showing expected clearance within the liver, bladder and kidneys and notable absence from the brain. (D) Whole mouse heart from a wild-type (WT) mouse injected from 3 days prior with mCntn2-800, in anterior-posterior (AP) and right lateral views. Atria outlined in white and cardiac chambers listed. LA, left atrium; LV, left ventricle; RA, right atrium; RV, right ventricle. Top: Brightfield. Bottom: NIR signal demonstrating labelling of the CCS (Blue->Red=Lowest->Highest signal). Mean signal to background ratio (SBR) as indicated. (E) Measured intervals (in msec) from sedated surface electrocardiograms (ECGs) including PR, QRS, QTc and RR in WT mice prior to (Day 0=baseline) and daily (Day 1, Day 2=post-injection) following a single tail vein injection of mCntn2-800. Intervals on a given day post-injection were compared to each mouse's pre-injection control baseline (Day 0) using a two-tailed, unpaired Student's t test. Significance considered at p<0.05. (F-H) Heart sections from a WT mouse injected 2 days prior with mCntn2-800. CCS components labelled with mCntn2-800 (Purple) and co-stained with known markers of the CCS including anti-Hcn4 (Red, SAN and His) or anti-Cx40 (Green, PF). DAPI (Blue, nuclei). LBB/RBB, left and right bundle branches; His, His bundle; PF, Purkinje fibers; SAN, sinoatrial node.

FIG. 2. Optical Clearing and 3D Volumetric Analyses on an Intact Heart Following mCntn2-800 Systemic Injection Reveals High Resolution Labeling of the Entire CCS. Schematic representation of workflow for iDISCO+ clearing of mouse hearts and visualization using light sheet microscopy. (B) iDISCO+ cleared heart harvested from a wild-type (CD1) mouse injected two days prior with mCntn2-800 (75 ug). Heart shown in three different angles of view including anterior-posterior (AP), right lateral (RL) and posterior-anterior (PA). Top and bottom rows are the same optically cleared heart using iDISCO+ where, in the top row, background fluorescence has been saturated to provide a representation of the opacified heart. Bottom row demonstrates the same tissue-cleared heart, showing near-infrared (800 nm) signal from mCntn2-800 marking the entire CCS. Conduction system components are labelled as indicated. AVN, atrioventricular node; His, His bundle; INT, internodal tracks; LA/RA, left or right atrium; LAVRB, left AV ring bundle; LBB/RBB, left of right bundle branch; LV/RV, left or right ventricle; PF, Purkinje fibers; RAVRB, right AV ring bundle; SAN, sinoatrial node.

FIG. 3. Fab Successfully Targets Alternative Cargo to the CCS. (A) Human anti-CNTN2 Fab antibody was biotinylated and conjugated to streptavidin-linked Saporin, a cell toxin (hCNTN2-Sap). (B) Wild-type mice received a single tail-vein injection of either hCNTN2-Sap (100 ug) (n=6) or Control-Sap (100 ug nonspecific human IgG similarly conjugated to Saporin) (n=6). Mice received daily electrocardiograms (ECGs) on Day 0 (baseline) and daily following injection with control or hCNTN2-Sap. On Day 2, hearts were harvested, fixed and immunostained. (C) Representative ECG tracings. (D) By Day 2 mice injected with hCNTN2-Sap demonstrated significant conduction abnormalities including prolonged PR, QRS and RR intervals as compared to mice injected with Control-Sap. Mean intervals with standard deviation. Gray bar, QTc interval corrected for QRS intervals. Statistical analyses using a two-tailed, unpaired Student's t test. Significance considered at p<0.05. (E) Consistent with targeted cell death, immunofluorescence of the CCS (red) showed subtotal loss of CCS cells as shown within the His bundle (His), right and left bundle branches (RBB/LBB) as stained by anti-Cntn2 staining. His, His bundle; IVS, interventricular septum; LBB, left bundle branch; RBB, right bundle branch.

FIG. 4. scRNA Sequence Analyses Reveal Novel Cell Surface Markers Within Distinct Components of the CCS For Generating Targeted Optical Imaging Tools. (A) Workflow of single-cell RNA sequencing (scRNAseq) analyses to uncover cell surface genes enriched within the murine CCS subcomponents including the sinoatrial node (SAN), atrioventricular node (AVN), His bundle (His) and Purkinje fiber (PF) cells, as compared to all other cardiac cell types. (B) Immunofluorescence (IF) staining of wild-type murine, embryonic day 16.5 cardiac tissue sections. Distinct CCS components shown including the sinoatrial node (SAN), atrioventricular node (AVN), His bundle (His), bundle branches (BB) and Purkinje fiber (PF) cells (each component outlined by a solid line) for two validated gene markers, Gfra2 and Epha4. DAPI (blue) in all images. (Top panel) IF with staining against Gfra2 protein (cyan) and known markers including Hcn4 (SAN, red), Cx40 (His, BB and PF, green), and Pgp9.5 (neurons, green or red as indicated). Transitional cells demarcated by hashed lines. (Bottom panel) IF with staining against Epha4 protein (cyan) and known markers including Hcn4 (SAN, red), Cpne5 (AVN, red), and Cx40 (His, BB and PF, green). cSAN, compact SAN; INT, internodal tract; IVS, interventricular septum; MV, mitral valve; LA, left atrial myocardium; LBB, left bundle branch; LV, left ventricle; PMJ, Purkinje-myocyte junction; Prox, proximal; RA, right atrial myocardium; RBB, right bundle branch; RBC, red blood cells; SAN Tz, SA nodal transitional cells; VM, ventricular myocardium.

FIG. 5. Nptn is Enriched Throughout the Murine and Human CCS. Immunofluorescence staining of wild-type, postnatal day 7 mouse (A-D) and 11 month old human (E-H) cardiac tissue sections. Mouse CCS (A-D): Anti-Nptn protein staining (cyan) within the (A) sinoatrial node (SAN) marked by Hcn4 (red); (B) atrioventricular node (AVN) labelled by Cpne5 (red); (C) His bundle (His), right bundle branch (RBB) and left bundle branch (LBB) indicated by Cpne5 (red); and (D) Purkinje fibers marked by Cx40 (red). DAPI (blue) in all panels. Human CCS (E-H): Anti-NPTN (orange) specifically staining the (A) SAN; (B) AVN; (C) His and LBB); and (D) Purkinje fibers (PF) co-stained with connexin 40 (CX40). DAPI (blue). INT, internodal tracts; IVS, interventricular septum; LV, left ventricle; MV, mitral valve; Prox, proximal; RA, right atrial myocardium; VM, ventricular myocardium.

FIG. 6. Systemic Injection of mNntn-800 in Mice Safely Labels the CCS In Vivo. (A) Experimental work flow. (B) Whole hearts from a wild-type (WT) mouse injected 1 day prior with mNptn-800 or IgG-800. Heart shown in posterior-anterior (PA) and right lateral (RL) views. Atria outlined in white and cardiac chambers listed. LA, left atrium; LV, left ventricle; RA, right atrium; RV, right ventricle. Top: Brightfield. Bottom: Near infrared (NIR) signal demonstrating labelling of the CCS (Blue->Red=Lowest->Highest signal). Mean signal to background ratio (SBR) as indicated. Bar graph showing mean SBR in hearts exposed to mNptn-800 (150 ug) or IgG-800 (Control). (C-D) Sedated surface electrocardiograms (ECGs) with measured intervals (in msec) including PR and QRS in WT mice prior to (Day 0=baseline) and daily (Day 1, Day 2, Day 3=post-injection) following a single tail vein injection of mNptn-800 (150 ug). n=3 for all time points. Intervals on a given day post-injection were compared to each mouse's pre-injection control baseline (Day 0) using a two-tailed, unpaired Student's t test. Significance considered at p<0.05. (E) Whole-body biodistribution of other tissue types, showing expected clearance within the liver and kidneys. (F-I) Heart sections from adult mouse injected 1 day prior with mNptn-800. (F) Compact SAN (cSAN) labelled by mNptn-800 signal (red) and absence of Cx40 (green) as opposed to Cx40+ right atrial myocardium (RA). (G) AVN labeled with mNptn-800 (red) and consistently lacking Cx40 (green) expression. His bundle (His) and proximal bundle branch (Prox BB); and, (I) Purkinje fibers (PF) co-stained with Cx40 (green) and mNptn-800 signal (red). mNptn-800 signal amplified using an anti-sheep 555 nm secondary antibody following tissue fixation. DAPI (Blue, nuclei). IVS, interventricular septum; VM, ventricular myocardium.

FIG. 7 (Supplemental FIG. 1). Cntn2-800 Dosage and Timecourse Analyses. (A) Experimental work flow for dosage analysis. Whole hearts from a wild-type (WT) mouse injected 3 days prior with polyclonal Cntn2-800 or non-specific IgG-800 (Control) at listed dosages. n=sample size per dose. Representative heart images with near infrared (NIR) signal demonstrating labelling of the CCS (Blue->Red=Lowest->Highest signal). (B) Bar graph showing a significant increase in mean signal to background ratio (SBR) at each dosage as compared to control IgG SBR using a two-tailed, unpaired Student's t test. Significance considered at p<0.05. (C) Work flow of time course analysis. Each WT mouse received a single 75 ug injection of Cntn2-800 and hearts were harvested at Day 1, 2, 3 or 4 for NIR imaging. n=3 mice per timepoint. (D) Bar graph showing no significant change mean SBR even out to 4 days post-injection to Day 1 following injection.

FIG. 8. (Supplemental FIG. 2). Human Anti-CNTN2 Monoclonal Fab Antibody Targets the CCS. (A) Titration ELISA with serial dilution of Fab. (B) SDS-polyacrylamide gel electrophoresis of purified Fab in reduced (˜25 kDa) and unreduced (˜50 kDa) states. (C) Human Fab-dye conjugate (hCNTN2-800). (D) Wild-type mice received a single tail-vein injection of hCNTN2-800 (150 ug). After 1 day, hearts were harvested, fixed and sectioned for fluorescence staining. (E) Purkinje fibers (PF) labelled by hCNTN2-800 signal (red) and co-stained with Cx40 (green). DAPI (Blue, nuclei). NIR, near-infrared.

FIG. 9 (Supplemental FIG. 3). Anti-CNTN2-Saporin Successfully Induces Targeted Cell Death of Major CCS Components. Immunofluorescence of the CCS (red, anti-Cntn2 immunostaining) showed subtotal loss of CCS cells within the sinoatrial node (SAN) and atrioventricular node (AVN) in wild-type mice injected 48 hours prior with a single tail-vein injection of either Control-Sap (100 ug nonspecific human IgG similarly conjugated to Saporin) (n=6) or hCNTN2-Sap (100 ug) (n=6).

FIG. 10 (Supplemental FIG. 4). Novel Cell Surface Markers Label Distinct Components of the Murine CCS. Fluorescence RNA in situ hybridization (FISH) staining of wild-type murine, embryonic day 16.5 cardiac tissue sections. Distinct CCS components shown including the sinoatrial node (SAN), atrioventricular node (AVN), His bundle (His), bundle branches (BB) and Purkinje fiber (PF) cells (each component outlined by a solid line) for each gene marker. DAPI (blue) in all images. (A, D) FISH targeting the known SAN marker Smoc2 or AVN/His/BB/PF marker Cpne5 (red punctae) as well as novel markers Pcdh17 (A) or Slc22a1 (D) (cyan punctae). (B-C) FISH targeting the known CCS marker Hon4 (red punctae) and known markers Slitrk5 (B) or Slit2 (C) (cyan punctae) mRNA. cSAN, compact SAN; INT, internodal tract; IVS, interventricular septum; MV, mitral valve; LA, left atrial myocardium; LBB, left bundle branch; LV, left ventricle; Prox, proximal; RA, right atrial myocardium; RBB, right bundle branch; RBC, red blood cells; SAN Tz, SA nodal transitional cells; VM, ventricular myocardium.

FIG. 11 (Supplemental FIG. 5). NPTN is Enriched in the CCS of the Adult Human Heart. Immunofluorescence staining of anti-NPTN (red) in cardiac tissue sections from a 45 year-old human heart showing (A) the compact sinoatrial node (cSAN) with inlays (a′) showing magnified border between surrounding right atrial myocardium (RA). (B) His bundle (His) and bundle branch (BB) with inlays (b′ and b″) showing magnified border region between His bundle (b′) or the bundle branch (b″) and the surrounding interventricular septum (IVS). DAPI (blue). INT, internodal tracts.

FIG. 12 (Supplemental FIG. 6). ECG intervals are Unchanged in Mice Injected with Nptn-800. Sedated surface electrocardiograms (ECGs) with measured intervals (in msec) including RR and QTc (Bazett's corrected QT interval) in wild-type mice prior to (Day 0=baseline) and daily (Day 1, Day 2, Day 3=post-injection) following a single tail vein injection of Nptn-800 (150 ug) in each animal. N=3 for all time points. Intervals on a given day post-injection were compared to each mouse's pre-injection control baseline (Day 0) using a two-tailed, unpaired Student's t test. Significance considered at p<0.05.

FIG. 13 depicts supplemental table 1.

FIG. 14 depicts supplemental table 2.

FIG. 15 depicts supplemental table 3.

FIG. 16 depicts supplemental table 4.

FIG. 17 depicts supplemental table 5.

FIG. 18 depicts supplemental table 6.

### DETAILED DESCRIPTION

This disclosure relates to theranostic tools for visualizing in real time the cardiac conduction system (CCS) or targeting drug delivery to the cardiac conduction system (CCS). Disclosed herewith reagents, compositions and methods may be helpful in preventing CCS damage due to surgical or catheter-based procedures. The reagents, compositions and methods may be also useful for targeting the CCS cells for therapeutic effects in patients, including patients with arrhythmia such as accelerated heart rhythm, heart block, or atrial or ventricular fibrillation. This disclosure provides antibodies, including antigen-binding fragments, that specifically bind to proteins of the CCS cells and reagents in which the antibodies are conjugated with a detection agent and/or a therapeutic drug. The disclosure also provides compositions comprising the reagents, methods for visualizing the CCS with the compositions, as well as methods for treating a subject, wherein a therapeutic drug is delivered to the subject's CCS by being conjugated with the antibody.

The term “protein” can be used in this disclosure to refer to a full polypeptide as well as any peptide and/or a protein domain, or any protein fragment, e.g., an epitope which may be a conformation of several amino acids displayed at the cell membrane surface of the CCS cell. The term “protein” further includes modified proteins, e.g., glycoproteins, and peptides. The term “protein” may be used interchangeably with the term “polypeptide.” Human Contactin 2 (CNTN2) is a protein, preferably having an amino acid sequence with SEQ ID NO: 9. Contactin 2 is encoded by the CNTN2 gene in humans. Synonyms for “CNTN2” which can be used interchangeably in this disclosure include, but are not limited to, CNTN2, AXT, DKFZp781D102, FLJ37193, FLJ2746, MGC157722, TAG-1, TAX, TAX1 and Contactin-2.

Human Neurotrimin (NTM) is a protein preferably having an amino acid sequence with SEQ ID NO: 21 and is one of the most enriched cell surface markers for the CCS cells. NTM is a member of the IgLON (LAMP, OBCAM, Ntm) family of immunoglobulin (Ig) domain-containing glycosylphosphatidylinositol (GPI)-anchored cell adhesion molecules.

Human Neuroplastin (NPTN) is a protein preferably having an amino acid sequence with SEQ ID NO: 22 and is a surface marker for the CCS cells. NPTN is a type I transmembrane protein belonging to the Ig superfamily expressed within the central nervous system in the murine and human heart.

Regents and compositions according to this disclosure may comprise one or more antibodies specific to Human Contactin 2 protein, human Neurotrimin protein or human Neuroplastin protein when these proteins are displayed at the surface of the CCS cells. Preferably, the regents and compositions comprise an antibody having a specific binding activity to Human Contactin 2 protein with SEQ ID NO: 9.

In this disclosure, the term “antibody” is understood broadly. For a detailed description of “an antibody,” a person of skill is referred to “Cellular and Molecular Immunology, the 9^{th} Edition” by Drs. A. Abbas, A. Lichtman and S. Pillal; published by Elsevier. Antibodies may include immunoglobulins: IgA, IgD, IgE, IgG and IgM or any combinations.

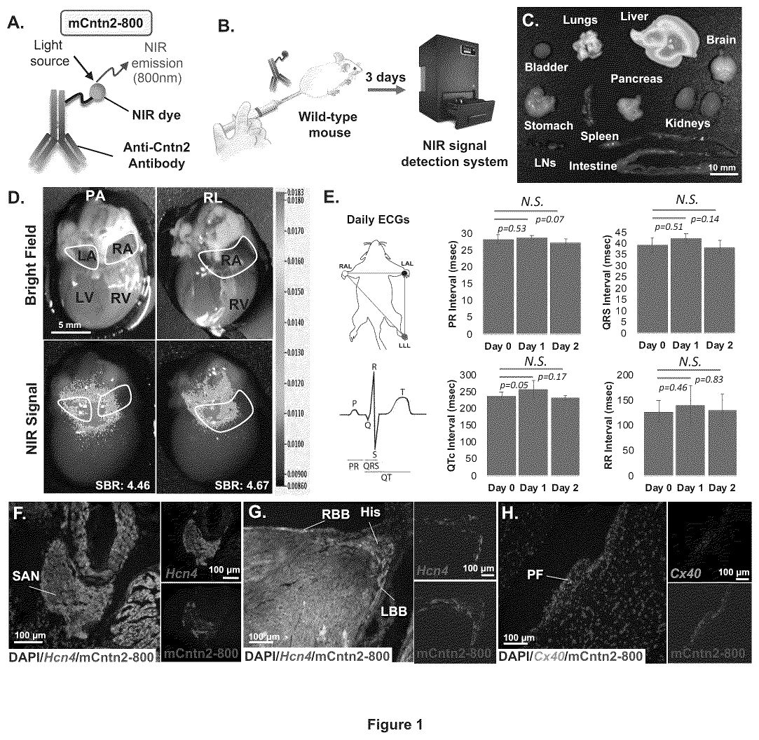

In embodiments, an antibody may comprise four polypeptides: two heavy chains, first heavy chain and a second heavy chain, and two light chains, a first light chain and a second light chain. The first heavy chain may be linked, preferably by a disulfide bond, to the first light chain and the second heavy chain may be linked, preferably by a disulfide bond, to the second light chain. In addition, the first heavy chain and the second heavy chain may be linked to each other, preferably with a disulfide bond. These linkages between the four polypeptides may result in formation of a “Y” shaped molecule, having two antigen-binding arms, each having an amino-terminal portion of the light chain linked to an amino-terminal portion of the heavy chain. In embodiments, the arms may be connected flexibly with a tether (the hinge region) to a stem which may be composed of only carboxy-terminal portions of the two heavy chains. One embodiment of a “Y” shaped antibody (and further conjugated with an NIR dye at the stem) is shown in FIG. 1A. The antibody stem may be referred to as Fc. Each of the antibody antigen-binding arms may be referred to as Fab (fragment, antibody binding). An antibody fragment having two arms may be referred to as F(ab)₂.

In this disclosure, each light chain polypeptide may comprise an amino-terminal light chain variable region (V_{L}) having from about 100 to about 130 amino acids. The light chain polypeptide may further comprise a carboxyterminal light chain constant region (C_{L}) or a portion thereof. Examples of light chain polypeptides include lambda (λ) and kappa (κ). Examples of suitable constant regions (C_{L}) include any constant regions as found for example, in lambda (λ) or kappa (κ) light chains. In some embodiments, the light chain polypeptide may include a variable region (V_{L}), but it does not include light chain constant region (C_{L}).

In this disclosure, each heavy chain polypeptide may comprise a heavy chain variable region (V_{L}), preferably composed from about 100 to about 130 amino acids. The heavy chain polypeptide may further comprise one to three heavy chain constant regions (C_{H1}, C_{H2} and C_{H3}) or at least a portion of the heavy chain constant region C_{H1}. Examples of heavy chain polypeptides include μ, δ, γ, α, and ε. Examples of suitable constant regions (C_{H1}) include any constant regions as found in μ, δ, γ, α, or ε heavy chains. In some embodiments, the heavy chain polypeptide may include a variable region (V_{L}), but it does not include heavy chain constant region (C_{H1}).

The variable regions (V_{L} and V_{H}) are located at the tip of the Y-shaped antibody arm and the specific amino acid sequence in the variable region determines the antibody specificity against an antigen.

The “functional” antibody fragment means that the fragment has a specific affinity an epitope. Accordingly, the functional antibody fragment may be used interchangeably with the term “antigen-binding fragment of the antibody.” In this disclosure, the antibody or its antigen-binding fragment may comprise at least one light chain polypeptide having at least a variable region (V_{L}) and at least one heavy chain polypeptide having at least a variable region (V_{H}), the light chain polypeptide being linked, e.g., by a disulfide bond or by a synthetic peptide linker, to the heavy chain polypeptide.

Examples of suitable antigen-binding fragments include, but are not limited to, a diabody (a dimer composed of variable region (V_{L}) linked to variable region (V_{H})), scFv, Fab and F(ab)₂. In this disclosure, scFv (single-chain variable fragment) refers to a fusion protein in which a variable region (V_{L}) of the light chain is fused in-frame with a peptide linker to a variable region (V_{H}) of the heavy chain.

In this disclosure, the antibody or its antigen binding fragment may be a recombinant antibody which may be obtained by screening a phage display library or by any other recombinant technology, or a monoclonal antibody obtained from a cell hybridoma, or a polyclonal antibody produced in a rabbit, goat, horse, chicken or any other species. The antibodies and antibody fragments may be at least partially or fully humanized. The antibodies include polyclonal, monoclonal, single-chain, and chimeric antibodies. Humanized monoclonal antibodies and their antigen-binding fragments are preferred.

In this disclosure, an antibody or a functional antibody fragment has a specific binding affinity to CNTN2 protein if the antibody or the functional (antigen-binding) antibody fragment has a binding constant, K_{d}, to the CNTN2 protein in the range from 10⁻⁶M to 10⁻¹² M. A person of skill will recognize that “K_{d}” stands for the equilibrium dissociation constant between an antibody and its epitope. In order to determine a K_{d}, a person of skill can follow a protocol disclosed in “Antibodies: a laboratory manual, Second Edition,” edited by E. Greenfield, 2014 or any other similar laboratory manuals generally available to a person of skill. Particularly preferred antibodies or antibody fragments include those with K_{d} to the CNTN2 epitope in the range from 10⁻⁹ M to 10⁻¹² M.

In this disclosure, “CDR” means a Complementarity Determining Region which is a part of the variable region (V_{L}) and the variable region (V_{H}). CDRs are amino acid domains which are about 6 to 20 amino acids long and which confirm specific recognition and binding to a particular protein antigen. A light chain variable region (V_{L}) may comprise three CDRs: CDR1, CDR2 and CDR3. A heavy chain variable region (V_{H}) may comprise at least two CDRs: CDR1, CDR2, with the third CDR3 being partially encoded by V_{H} and the rest of CDR3 being encoded by the DH gene segment and part of the JH gene segment. The variable regions, CDRs, may be spaced from each other by framework regions which may form β-sheets and provide the structural scaffolding in order to project CDR loops and make CDR loops accessible for interaction with an antigen epitope.

In this disclosure, preferred antibodies are defined by their amino acid sequences for CDR regions and/or light and heavy chain variable regions since these are amino acid sequences which define the specific recognition of the selected antigen, such as for example, as contactin 2 protein expressed at the surface of the CCS cells. These preferred antibodies may or may not further comprise at least a portion of at least one constant region with the proviso that any constant region or its portion typically found light or heavy antibody chains can be also used in some preferred antibodies according to this disclosure.

The antibody amino acid sequences may include amino acid variants having an amino acid sequence with from at least 70% to at least 99% amino acid sequence identity to the amino acid sequence with a SEQ ID NO. defined in the enclosed sequence listing, e.g., having at least 71%, 72%, between 73 and 80%, at least 80%, 81%, 82%, 83%, 84%, 85%, 86%, 87%, 88%, 89%, 90%, 91%, 92%, 93%, 94%, 95%, 96%, 97%, 98% or at least 99% amino acid sequence identity to an amino acid sequence defined with the SEQ ID NO. It will be appreciated that the variants may be referred to as functional variants because they have the same or similar specific binding activity to the same antigen as the (control) antibody for which the amino acid sequence is provided. “Similar” means±20% of the binding value for the control antibody.

In comparison to an amino acid sequence, its amino acid variant may include a conservative amino acid replacement which results in an amino acid replacement in a protein that changes a given amino acid to a different amino acid with similar biochemical properties (e.g., charge, hydrophobicity and size) or a replacement of amino acids which does not change substantially the binding of the antibody to its antigen. In particular, amino acid replacements may be introduced primarily into one or more of the framework regions, while the CDRs that control selective recognition of an antigen may remain intact or substantially intact, meaning that only one or two amino acids may be modified in the CDR, if at all.

In this disclosure, nucleic acid sequences include variants having a nucleic sequence with at least 70% to at least 99% nucleic acid sequence identity to the nucleic acid sequence with a SEQ ID NO. defined in the enclosed sequence listing, e.g., having at least 71%, 72%, between 73 and 80%, at least 80%, 81%, 82%, 83%, 84%, 85%, 86%, 87%, 88%, 89%, 90%, 91%, 92%, 93%, 94%, 95%, 96%, 97%, 98% or at least 99% nucleic acid sequence identity.

It will be appreciated that the nucleic acid variants encode polypeptides that have the same or similar specific binding activity to the same antigen as the (control) antibody for which the amino acid sequence is provided. “Similar” means±20% of the binding value for the control antibody.

Nucleic acid variants may include silent mutations that do not result in changes of an amino acid sequence, codon optimization for a particular species in which the nucleic acid is to be expressed and/or substitutions that result in a conservative amino acid replacement which results in an amino acid replacement in a protein that changes a given amino acid to a different amino acid with similar biochemical properties (e.g., charge, hydrophobicity and/or size), or any other replacement of amino acids which does not change the binding of the antibody to its antigen. In particular, nucleic acid substitutions may be introduced primarily into one or more of the framework regions, while portions encoding CDRs that control selective recognition of an antigen may remain intact or substantially intact.

In one aspect, this disclosure provides an antibody or an antigen-binding fragment thereof, having a specific binding activity to human contactin 2 (CNTN2) protein with the amino acid sequence of SEQ ID NO: 9, the antibody or the antigen-binding fragment thereof comprising:
a). at least a first polypeptide comprising a light chain variable region (V_{L}); and
b). at least a second polypeptide comprising a heavy chain variable region (V_{H}),
wherein the first polypeptide is linked to the second polypeptide,
wherein the light chain variable region (V_{L}) of the first polypeptide comprises at least one from the following four amino acid sequences:
   the amino acid sequence with SEQ ID NO: 1 or a variant thereof with at least 70% amino acid sequence identity to SEQ ID NO: 1;
   the amino acid sequence with SEQ ID NO: 5 or a variant thereof with at least 70% amino acid sequence identity to SEQ ID NO: 5;
   an amino acid sequence comprising Complementarity-Determining Region 1 (CDR1) with SEQ ID NO: 10 or a variant thereof with at least 90% amino acid sequence identity to SEQ ID NO: 10, Complementarity-Determining Region 2 (CDR2) with SEQ ID NO: 11 or a variant thereof with at least 90% amino acid sequence identity to SEQ ID NO: 11, and Complementarity-Determining Region 3 (CDR3) with SEQ ID NO: 13 or a variant thereof with at least 90% amino acid sequence identity to SEQ ID NO: 13; or
   an amino acid sequence comprising CDR1 with SEQ ID NO: 15 or a variant thereof with at least 90% sequence identity to SEQ ID NO: 15, CDR2 with SEQ ID NO: 16 or a variant thereof with at least 90% sequence identity to SEQ ID NO: 16, and CDR3 with SEQ ID NO: 17 or a variant thereof with at least 90% sequence identity to SEQ ID NO: 17; and
wherein the heavy chain variable region (V_{H}) of the second polypeptide comprises at least one from the following four amino acid sequences:
   an amino acid sequence with SEQ ID NO: 3 or a variant thereof with at least 70% amino acid sequence identity to SEQ ID NO: 3;
   an amino acid sequence with SEQ ID NO: 7 or a variant thereof with at least 70% amino acid sequence identity to SEQ ID NO: 7;
   an amino acid sequence comprising CDR1 with SEQ ID NO: 13 or a variant thereof having at least 90% sequence identity to SEQ ID NO: 13, and CDR2 with SEQ ID NO: 14 or a variant thereof with at least 90% identity to SEQ ID NO: 14; or
   an amino acid sequence comprising CDR1 with SEQ ID NO: 18 or a variant thereof with at least 90% sequence identity to SEQ ID NO: 18, and CDR2 with SEQ ID NO: 19 or a variant thereof with at least 90% sequence identity to SEQ ID NO: 19.

Suitable antibodies may include antibodies and antibody fragments in which the first polypeptide is linked, preferably by a disulfide bond and/or via a peptide linker, to the second polypeptide. Certain antibodies and antibody fragments may only include the first polypeptide and the second polypeptide. In some preferred embodiments, the first polypeptide and/or the second polypeptide may not comprise a constant region.

In some embodiments, the antibody or the antigen-binding fragment may further comprise a third polypeptide comprising the light chain variable region (V_{L}), the third polypeptide being linked to a fourth polypeptide, the fourth polypeptide comprising the heavy variable region (V_{H}), and wherein the second polypeptide and the fourth polypeptide may be linked. Suitable antibodies include those which do not comprise the stem region Fc.

In some embodiments, the antibody or the antigen-binding fragment thereof may include those, wherein the first polypeptide further comprises at least a portion of a light chain constant region (C_{L}) and wherein the second polypeptide further comprises a least a portion of a heavy chain constant region (C_{H1}). Preferred light chain constant regions (C_{L}) include those as can be found in lambda (λ) or kappa (κ) light chains. Preferred heavy chain constant regions (CH) include those as can be found in μ, δ, γ, α, and ε polypeptides.

The preferred antibody or the antigen-binding fragment includes a human monoclonal fragment antigen-binding region (Fab) composed of the first polypeptide a). linked to the second polypeptide b)., wherein the first polypeptide further comprises at least a portion of a light chain constant region (C_{L}) and wherein the second polypeptide further comprises a least a portion of a heavy chain constant region (C_{H1}).

Particularly preferred antibodies further include the antibody or the antigen-binding fragments, wherein the first polypeptide may comprise the amino acid sequence with SEQ ID NO: 1 or 5, or a variant amino acid sequence with at least 90% or at least 95% amino acid sequence identity to SEQ ID NO: 1 or 5; and wherein the second polypeptide may comprise the amino acid sequence with SEQ ID NO: 3 or 7, or a variant amino acid sequence with at least 90% or at least 95% amino acid sequence identity to SEQ ID NO: 3 or 7.

The following underlined sequences are CDR1, CDR2 and CDR3, respectively, spaced with the framework regions (not underlined) in the light chain variable region (V_{L}) with SEQ ID NO: 1.

(SEQ ID NO: 1)
EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPWT
FGQGTKVEIK

The following sequence is the nucleic acid with SEQ ID NO: 2 encoding the light chain variable region (V_{L}) with SEQ ID NO. 1

(SEQ ID NO: 2)
GAAATTGTGTTGACACAGTCTCCAGGCACCCTGTCTTTGTCTCCAGGGG
AAAGAGCCACCCTCTCCTGCAGGGCCAGTCAGAGTGTTAGCAGCAGCTA
CTTAGCCTGGTACCAGCAGAAACCTGGCCAGGCTCCCAGGCTCCTCATC
TATGGTGCATCCAGCAGGGCCACTGGCATCCCAGACAGGTTCAGTGGCA
GTGGGTCTGGGACAGACTTCACTCTCACCATCAGCAGACTGGAGCCTGA
AGATTTTGCAGTGTATTACTGTCAGCAGTATGGTAGCTCACCTTGGACG
TTCGGCCAAGGGACCAAGGTGGAAATCAAAC

The following underlined sequences are CDR1 and CDR2, respectively, spaced with the framework regions (not underlined) in the heavy chain variable region (V) with SEQ ID NO: 3.

(SEQ ID NO: 3)
QVHLVESGGGLLQPGGSLRLSCAASGFTFSSYAMSWVRQAPGKGLEWVS
GIVGNGGSTYYADSVKGRFTISRDNSKNTLYLQMNSLRAEDTAVYYCAK
LEWGALGYWGQGTLVTVSS

The following sequence is the nucleic acid with SEQ ID NO: 4 encoding the heavy chain variable region (V_{H}) with SEQ ID NO. 3

(SEQ ID NO: 4)
CAGGTGCACCTGGTGGAGTCTGGGGGAGGCTTGCTACAGCCTGGGGGGT
CCCTGAGACTCTCCTGTGCAGCCTCTGGATTCACTTTTAGCAGCTATGC
CATGAGCTGGGTCCGCCAGGCTCCAGGGAAGGGGCTGGAGTGGGTCTCA
GGTATTGTTGGTAATGGTGGTAGCACATACTACGCAGACTCCGTGAAAG
GCCGGTTCACCATCTCCAGAGACAATTCCAAGAACACACTGTATCTGCA
AATGAACAGCCTGAGAGCCGAGGACACCGCCGTATATTACTGTGCGAAA
TTGGAGTGGGGGGCCTTGGGGTACTGGGGCCAGGGAACCCTGGTCACCG
TCTCAAGC

The following underlined sequences are CDR1, CDR2 and CDR3, respectively, spaced with the framework regions (not underlined) in the light chain variable region (V_{L}) with SEQ ID NO: 5.

(SEQ ID NO: 5)
DVVMTQSPLSLPVTLGQPASISCRSSQSLVYSDGNTYLYWFQQRPGQSP
RRLIYQVSNRDSGVPDRFSGSGSGTDFTLKISRVEAEDVGVYYCMQALE
TPITFGQGTRLEIK

The following sequence is the nucleic acid with SEQ ID NO: 6 encoding the light chain variable region (V_{L}) with SEQ ID NO. 5

(SEQ ID NO: 6)
GATGTTGTGATGACTCAGTCTCCACTCTCCCTGCCCGTCACCCTTGGAC
AGCCGGCCTCCATCTCCTGCAGGTCTAGTCAAAGCCTCGTATACAGTGA
TGGAAACACCTACTTGTATTGGTTTCAGCAGAGGCCAGGCCAATCTCCA
AGGCGCCTAATTTATCAGGTTTCTAACCGGGACTCTGGGGTCCCAGACA
GATTCAGCGGCAGTGGGTCAGGCACTGATTTCACACTGAAAATCAGCAG
GGTGGAGGCTGAGGATGTTGGGGTTTATTACTGCATGCAAGCTCTAGAA
ACTCCGATCACCTTCGGCCAAGGGACACGACTGGAGATTAAAC

The following underlined sequences are CDR1 and CDR2, respectively, spaced with the framework regions (not underlined) in the heavy chain variable region (V_{H}) with SEQ ID NO: 7.

(SEQ ID NO: 7)
EVQLVETGGGLIQPGGSLRLSCAASGFTVSSNYMSWVRQAPGKGLEWVS
VIYSGGSTYYADSVKGRFTISRDNSKNTLYLQMNSLRAEDTAVYYCARD
RIAAAGTLPVYWGQGTLVTVSS

The following sequence is the nucleic acid with SEQ ID NO: 8 encoding the heavy chain variable region (V_{H}) with SEQ ID NO. 7

(SEQ ID NO: 8)
GAGGTGCAGCTGGTGGAGACCGGAGGAGGCTTGATCCAGCCTGGGGGGT
CCCTGAGACTCTCCTGTGCAGCCTCTGGGTTCACCGTCAGTAGCAACTA
CATGAGCTGGGTCCGCCAGGCTCCAGGGAAGGGGCTGGAGTGGGTCTCA
GTTATTTATAGCGGTGGTAGCACATACTACGCAGACTCCGTGAAGGGCC
GATTCACCATCTCCAGAGACAATTCCAAGAACACGCTGTATCTTCAAAT
GAACAGCCTGAGAGCCGAGGACACGGCCGTGTATTACTGTGCGAGAGAC
CGTATAGCAGCAGCTGGTACCCTTCCCGTCTACTGGGGCCAGGGCACCC
TGGTCACCGTCTCAAGC

Various methods can be used for producing the antibodies or antibody fragments according to this disclosure. In some preferred embodiments, human contactin 2 protein with SEQ ID NO: 9 or at least a portion thereof, preferably the portion shown as underlined sequence below, can be used for screening a phage-display library, for example by following a protocol provided in Example 7.

| (SEQ ID NO: 9) | |
|---|---|
| 1 | mgtatrrkph lllvaavalv sssawssalg sqttfgpvfe dqplsvlfpe esteeqvlla |
| 61 | crarasppat yrwkmngtem klepgsrhql vggnlvimnp tkaqdagvyq clasnpvgtv |
| 121 | vsreailrfg flqefskeer dpvkahegwg vmlpcnppah ypglsyrwll nefpnfiptd |
| 181 | grhfvsqttg nlyiartnas dlgnysclat shmdfstksv fskfaqlnla aedtrlfaps |
| 241 | ikarfpaety alvgqqvtle cfafgnpvpr ikwrkvdgsl spqwttaept lqipsvsfed |
| 301 | egtyeceaen skgrdtvqgr iivqaqpewl kvisdteadi gsnlrwgcaa agkprptvrw |
| 361 | lrngeplasq nrvevlagdl rfsklsleds gmyqcvaenk hgtiyasael avqalapdfr |
| 421 | lnpvrrlipa arggeilipc qpraapkavv lwskgteilv nssrvtvtpd gtliirnisr |
| 481 | sdegkytcfa enfmgkanst gilsvrdatk itlapssadi nlgdnltlqc hashdptmdl |
| 541 | tftwtlddfp idfdkpgghy rrtnvketig dltilnaqlr hggkytcmaq tvvdsaskea |
| 601 | tvlvrgppgp pggvvvrdig dttiqlswsr gfdnhspiak ytlqartppa gkwkqvrtnp |
| 661 | aniegnaeta qvlgltpwmd yefrviasni lgtgepsgps skirtreaap svapsglsgg |
| 721 | ggapgelivn wtpmsreyqn gdgfgyllsf rrqgsthwqt arvpgadaqy fvysnesvrp |
| 781 | ytpfevkirs ynrrgdgpes ltalvysaee eprvaptkvw akgvsssemn vtwepvqqdm |
| 841 | ngillgyeir ywkagdkeaa adrvrtagld tsarvsglhp ntkyhvtvra ynragtgpas |
| 901 | psanattmkp pprrppgnis wtfsssslsi kwdpvvpfrn esavtgykml yqndlhltpt |
| 961 | lhltgknwie ipvpedigha lvqirttgpg gdgipaevhi vrnggtsmmv enmavrpaph |
| | pgtvishsva mliligslel |

In another aspect, this disclosure relates to reagents useful for therapeutic, imaging and/or diagnostic applications. The reagents may comprise the one or more antibody or the antibody fragments according to this disclosure and wherein the one or more antibody or the antibody fragments are conjugated to a detection agent and/or a therapeutic drug. In this disclosure, the reagents include therapeutic products suitable for treating patients. In this disclosure, compositions may include one or more reagents. The detection agent and/or the therapeutic drug may be conjugated with the antibody or the antibody fragment directly or indirectly.

In this disclosure “conjugated directly” means that one or more moieties, e.g., an imaging dye, is covalently attached (linked) or non-covalently attached (linked) to the antibody or the functional antibody fragment.

In this disclosure, “conjugated indirectly” means that the moiety, e.g., a therapeutic drug (small organic molecule or a biomolecule) or a dye (diagnostic or detection agent), is attached to the antibody (or its antigen binding fragment) via a linker, which may be a synthetic peptide, biotin or streptavidin. In the alternative, the therapeutic drug or the detection agent may be encapsulated in a carrier, e.g., a liposome or some other type of a carrier and/or shell, and the antibody or the antigen binding fragment thereof may be displayed at the surface of the carrier and targets the carrier (along with the drug and/or dye) to the CCS cells. In some of these embodiments, the antibody or its antigen binding fragment may be modified by linking in frame to its carboxyterminal portion a moiety, e.g., a transmembrane domain or a hydrophobic chemical moiety for anchoring the antibody or its antigen binding fragment to the liposomes or other carriers.

In this disclosure, the term “linker” may refer to any molecule, e.g., a short peptide or any other group of atoms or a chemical molecule which is positioned between the antibody or its fragment and the therapeutical drug and/or detection agent. Preferably, the linker is linked with at least one of its atoms or residues to the antibody or its fragment. The linker is also linked with at least one of its atoms or residues to the therapeutic drug, diagnostic agent or a carrier. Accordingly, the linker links the antibody and the drug and/or the detection agent together.

The “detection agents” of this disclosure may include radioactive isotopes, nanoparticles, biotin, tags, e.g., the histidine tag, or imaging agents. A great variety of imaging agents is currently available. Preferred imaging agents in this disclosure include contrast agents and MRI dyes which are typically used in clinical procedures. Particularly preferred imaging agents include those with biocompatible NIR (near-infrared) fluorophore.

Through the standard NHS ester chemistry (NHS ester reactive group provides the functionality for labeling primary and secondary amines, such as lysine residues in proteins), or maleimide chemistry (functionality for labeling molecules that contain free sulfhydryl (—SH) groups, such as cysteine residues in proteins), or other forms of conjugation chemistry, the antibodies or their antibody fragments of this disclosure may be covalently or non-covalently conjugated to any standard dyes of the visible and non-visible spectrums (e.g. fluorescein, 488, FITC, 555, Cy3, Cy5, Cy5.5, 598, 640, 680, 780, 800, zw800, indocyanine green, 900 and newly designed and ultrapurified fluorescent probe-antibody conjugates with fluorescence emissions in the NIR-II region 1,000-1,700 nm). Similarly, additional conjugates for therapeutic and diagnostic use may include contrast agents (CT and MRI), radiotracers, quantum dots and/or nanoparticles, preferably superparamagnetic iron oxide nanoparticles (SPION) or gold nanoparticles. Particularly preferred imaging dyes for the reagents and compositions of this disclosure may include LiCor IRDye 800CW which are near-infrared fluorescent dyes available from LiCor BioSciences U.S.

The present disclosure provides a CCS imaging and/or diagnostic reagent which may comprise, consist essentially of, or consist of the antibody or its antigen binding fragment being conjugated to one or more detection agents, and the CCS imaging and/or diagnostic reagent capable of binding to a cell of the cardiac conductions system (CCS), and wherein the antibody has a binding affinity to a CCS cell surface marker displayed at the outer surface of the CCS cell membrane. Preferably, the marker is human contactin 2 protein, neurotrimin protein or neuroplastin protein. The detection agent may be an imaging dye comprising a chromophore, a fluorophore, a tag, a radioactive isotope, a small molecule, a biomolecule, and/or a nanoparticle. Any of the CCS imaging and/or diagnostic reagents in this disclosure may be also referred as “an optical imaging agent.”

The disclosure also provides methods for preparing the CCS imaging and/or diagnostic reagents (optical imaging agents). These methods include linking the detection agent, e.g., an imaging dye, such as for example a biocompatible NIR dye to the antibody according to this disclosure. Once conjugated the final dye: protein ratio can be in the range 1:1000 to 1000:1. Particularly preferred reagents of this disclosure include those in which the antibody is conjugated to a detection agent which comprises a biocompatible NIR (near-infrared) fluorophore which can be an NIR dye with broad absorption spectrum (778 nm) and emission (795 nm). In the conjugates, the preferred dye: protein ratio may be 1.5-2.

In another aspect, the present disclosure provides a CCS therapeutic product which comprises, consists essentially of, or consists of the antibody or its antigen binding fragment being conjugated to one or more therapeutic drugs. The CCS therapeutic product (regent) may bind to a cell of the cardiac conductions system (CCS) since the antibody has a binding affinity to a CCS cell surface marker displayed at the outer surface of the CCS cell membrane. Preferably, the marker is human contactin 2, neurotrimin or neuroplastin.

Some of the compositions and therapeutic products (reagents) of this disclosure may comprise a therapeutic drug which may be a biomolecule or a small molecule. Suitable biomolecules include a DNA, an RNA molecule and/or CAS9 enzyme-single guide complex, or any other gene-editing molecules which alter expression and/or function of one or more genes and/or proteins in the CCS cell. Suitable RNA or DNA molecules, e.g. CAS9 enzyme-single guide complex, include those that can silence or activate the biology activity of the CCS cell.

For CCS therapeutic applications, the antibody or its antigen binding fragment may be conjugated with small molecule drugs such as digoxin, calcium channel blocker, beta blocker, or anti-arrhythmic drugs or RNA or DNA that can silence or activate at least one biologic function of the CCS cell. Some of the compositions and reagents of this disclosure may comprise the antibody or its antigen binding fragment conjugated with one or more of the following drugs: antiarrhythmics, CCS agonists, or anti-inflammatoires. Suitable antiarrhythmics include, but are not limited to, Class I drugs which block voltage-gated Na channels, Class II drugs which are β-Blockers, Class III drugs which prolong the action potential, usually via K+ channel blockade, or Class IV drugs which are Ca2+ antagonists. The following drugs are non-non-liming examples of suitable antiarrhythmics: Procainamide, Quinidine, Disopyramide, Lidocaine, phenytoin, tocainide, mexiletine, Flecainide, encainide, propafenone, moricizine, Atenolol, acebutolol, metoprolol, nadolol, propranolol, NAPA, Amiodarone, Sotalol, Ibutilide, Amlodipine, diltiazem, felodipine, isradipine, nicardipine, nifedipine, nisoldipine, verapamil, sodium/potassium/ATPase blocker (e.g. digoxin), and adenosine receptor blocker (e.g. adenosine, dipyridamole). CCS agonists include: beta 1 agonists: (e.g. dobutamine); beta 2 agonists: (e.g. salmeterol, terbutaline); and non-selective beta agonists: (e.g. isoproterenol, epinephrine). Antiinflammatories may include NSAIDs (e.g. ibuprofen, naproxen), preferential and selective COX2 inhibitors, corticosteroids (e.g. prednisone, methyl-prednisone), conventional DMARDS (e.g. methotrexate, leflunomide, sulfasalazine, hydroxychloroquine) and biologic DMARDs (e.g. Adalimumab, Etanercept, Infliximab, Rituximab, Abatacept).

Particularly preferred therapeutic drugs may also include one or more of the following: calcium channel blockers (e.g. diltiazem, verapamil), beta adrenergic blockers (e.g. metoprolol, carvediolol, atenolol), sodium/potassium/ATPase blockers (e.g. digoxin), adenosine receptor blockers (e.g. adenosine, dipyridamole), cytotoxic agents (e.g. diphtheria toxin A, methotrexate, doxorubicin), adrenergic agonists (e.g. isoproterenol, epinephrine) and anti-inflammatory (e.g. glucocorticoids, cyclosporin A, tacrolimus).

Particularly preferred reagents include those in which the antibody or its antigen binding fragment is conjugated with a near-infrared dye (NIR), a superparamagnetic iron oxide nanoparticle (SPION), a gold nanoparticle, saporin, an antiarrhythmic drug, a ribonucleic acid (RNA) or deoxyribonucleic acid (DNA), a CAS9 enzyme-single guide complex, amiodarone or procainamide.

In yet another aspect, this disclosure relates to compositions comprising the antibody or the antigen-binding fragment thereof according to this disclosure and/or the regent according to this disclosure, and one or more excipients.

In embodiments, the compositions may comprise one or more of the CCS cell-specific antibodies and one or more excipients which may be pharmaceutically acceptable excipients. The excipients may be water, a buffer and/or any other solvent, a filler, a bulking agent, and/or carrier. The compositions also include those with one or more excipients for oral, topical, local or systemic, e.g., intravenous (IV) injection, administration to a subject. These compositions may comprise from 0.1 wt % to 99.9 wt % of the CCS cell specific antibody, such as the CNTN2 specific monoclonal antibody or its antigen binding fragment according to this disclosure, and from 0.1 wt % to 99.9 wt % of the one or more excipients.

This disclosure also provides compositions which may comprise, consist essentially of, or consist of the reagent and one or more pharmaceutically acceptable excipients which may be water, a buffer and/or any other solvent, a filler, a bulking agent, and/or carrier. The compositions also include those in which one or more reagents are formulated with one or more excipients for oral, topical, local or systemic, e.g., intravenous (IV) injection, administration to a patient. These compositions may comprise from 0.1 wt % to 99.9 wt % of the reagent and from 0.1 wt % to 99.9 wt % of the one or more excipients.

The regents and compositions according to this disclosure may be suitable for visualizing the CCS in real time, e.g. during a surgical procedure. These regents and compositions may be also suitable for delivering therapeutic drugs selectively to the CCS region in a subject.

In yet another embodiment, this disclosure provides methods for treating a subject. The methods may comprise administering to the subject one or more antibodies or their antigen binding fragments according to this disclosure, one or more reagents according to this disclosure or one or more compositions according to this disclosure. The antibodies, reagents or compositions may be administered orally, topically, locally or systemically. The antibody or its antigen binding fragment, the regent or the composition may administer in any suitable amount, for example from about 0.05 mg to about 100 mg of the antibody, its antigen binding fragment, therapeutic drug and/or detection agent per one kilogram of the subject's body weight.

In some embodiments for application as CCS imaging agents in real-time, the antibody or its antigen binding fragment conjugated to any of the detection agents according to this disclosure may be given to a subject either topically or by intravenous or intracoronary systemic delivery. The administration of this composition delivers the detection agent to the CCS and the CCS can be then monitored in real time during a cardiac surgery or procedure with one or more devices typically used to detect the detection agent. Visualizing and monitoring the CCS in real time during the cardiac surgery may help with preventing iatrogenic damage to the conduction system during both congenital and adult heart surgeries. The CCS injuries can result in heart block, arrhythmias, decreased cardiac output, and even sudden death. Iatrogenic damage to the CCS remains a significant surgical complication in both congenital heart disease (CHD) and adult cardiac surgeries. This is due in part to an inability to visualize and, thus, avoid damaging the surrounding CCS by inadvertent incision or suture placement. In the current era, postoperative heart block alone, secondary to accidental surgical damage of the atrioventricular node, complicates roughly 1-3% of all CHD surgeries. Postoperative heart block and other forms of intraoperative CCS damage remain a significant cause of morbidity, increased cost, decreased long-term survival and often the life-long need for mechanical pacemakers.

The present visualization methods for the CCS in real time can be helpful in conjunction with various adult cardiac surgeries, including mitral and aortic valve replacements, myomectomy in hypertrophic cardiomyopathy. The ability to visualize and monitor the CCS in real time provides a significant technical contribution to the current standard of care in both pediatric and adult intracardiac surgeries.

The disclosed herewith compositions and methods can directly target the CCS structure in order to therapeutically modify the behavior of these cells. For example, the atrial-ventricular node (AVN) is the key structure that regulate the ventricular heart rate during atrial arrhythmia such as atrial fibrillation, a major disease order in aging population with significant healthcare expenditure. The ability to direct target the AVN to provide it with drugs that would reduce its ability to conduct electrical activity from the atria during atrial fibrillation represents a major advantage over the current use of drugs that blocks AVN which has also blood pressure lowering effects when patients are usually hypotensive. In addition, there is emerging data to support the origin of ventricular fibrillation from the Purkinje fiber network. Being able to target the Purkinfe fiber structure of the CCS with antibodies directly binding and delivery therapeutic agents to the Purkinje cells provides a major improvement in comparison with non-specific effects of anti-arrhythmic drugs that are currently being used.

Further applications for the antibody-imaging agent method according to this disclosure include other modes of imaging such as MRI or CT. For example, the near infrared dye may be replaced with other molecules (e.g., iron tag, magnetic beads, etc.) that can be detected by MRI, CT, or intracardiac catheters, etc.).

In some embodiments, methods may comprise visualizing the CCS in real time in a subject by administering to the subject one or more compositions of this disclosure which comprise the CCS imaging and/or diagnostic reagent of this disclosure. These methods may include cardiothoracic surgeries and catheter procedures. The routes of administration for the compositions in these methods include oral, topical, local or systemic. After the composition is administered to the subject, the CCS imaging and/or diagnostic reagent binds to CCS cells. The CCS in the subject is then visualized in real time with the detection agent which is bound to the CCS cells via the antibody to which the detection agent is conjugated. Thus, the CCS can be monitored in real time during diagnostic and/or surgical procedures. In these methods, detection may comprise illuminating the CCS with UV, visible, and/or infrared light with a scope and/or by directly shining the light at the CCS if this is an open-heart surgery. Detection may also comprise an ultrasound and computed tomography if the CCS is examined in connection with diagnostic tests which may include neonatal tests. The detection may further comprise capturing images of the CCS with a camera in real time and further storing the images of the CCS in a computer system, if necessary, and optionally transmitting the images to at least one remote location.

The disclosure also provides treatment methods which comprise administering to a subject one or more compositions of this disclosure which comprise the therapeutic product. The treatment methods include those for treating cardiac arrhythmia in a subject or any other the CCS related disorders and/or diseases, including accelerated heart rhythm, heart block, or atrial or ventricular fibrillation.

In present visualization/diagnostic and/or treatment methods, the compositions according to this disclosure may be administered orally, topically, locally or systemically to a subject in any suitable amount, as may be needed. Typically, from about 0.05 mg to about 100 mg of an active ingredient (the therapeutic drug) per one kilogram of the body weight may be administered. The dosages may be adjusted as needed, depending on various factors, including the subject's weight, his/her metabolic response to the medication.

Embodiments also include a method for visualizing the cardiac conduction system (CCS) in a subject, the method comprising:
administering to the subject the composition according to this disclosure, the composition comprising at least one contactin 2 specific antibody or its antigen-binding fragment, or administering to the subject a composition comprising one or more antibodies with a specific binding activity to human neurotrimin protein (NTM) or human neuroplastin protein (NPTN); and
visualizing the CCS in the subject in real time by contacting the subject with ultrasound, computed tomography, visible, UV and/or infrared light.

Iatrogenic damage to the CCS, which surrounds many key interventional targets (e.g. heart valves) during cardiac procedures, can result in decreased heart function and a host of irreversible, life-threatening arrhythmias (e.g. heart block), often requiring permanent pacemaker device placement. To address this unmet medical need this disclosure provides the antibodies, antigen binding fragments, the reagents and the compositions that allow for the real-time visualization of the CCS during invasive procedures with high sensitivity, specificity and resolution. The antibodies and its antigen-binding fragments as defined in this disclosure target the CCS with high specificity.

These reagents and compositions provide several technical advantages, including: 1) high specificity and spatial resolution; 2) lack of additional technical expertise needed to visualize the CCS; and 3) real-time visualization without disruption of the surgical field or workflow. Integration of optical imaging diagnostic tools into the surgical management of cardiothoracic surgery has the potential to dramatically improve adverse outcomes in both pediatric and adult cardiac surgeries. Specifically, the inventors envision that high-resolution, real-time CCS detection through direct, intraoperative visualization by surgeons will minimize the risk of iatrogenic damage, thereby reducing hospital costs and length of stay as well as the need for lifelong pacemaker dependency and overall morbidity and mortality associated with cardiac surgeries.

Importantly, this approach additionally lends itself to the broader potential of molecular targeting of the CCS in vivo, including systemic delivery of other forms of cargos such as contrast agents or therapeutics. Use of advanced imaging, including cardiac MRI and CT have become a critical aspect of preprocedural planning in invasive cardiac interventions.

Beyond the surgical suite, damage to the CCS is also a significant risk during invasive catheter-based procedures such as cardiac electrophysiology ablations, for which there are ˜40,000 adult cases per year in the U.S. alone. Pre-procedural mapping of the CCS using the regents, compositions and methods according to this disclosure may provide at least some of the following technical advantages: 1) Improving ablation outcomes through direct visualization of target structures (e.g. slow pathway conduction fibers that serve as the nidus of atrioventricular nodal re-entrant tachycardia); 2) Minimizing the risk of iatrogenic damage of critical CCS components (e.g. detailing the proximity of pathways targeted for ablation in relation to other critical CCS structures); and 3) Helping to guide pacemaker device placement (e.g. His bundle lead placement).

The most effective intravenous antiarrhythmics may include Amiodarone and Procainamide. However both may be associated with significant unwanted systemic side effects in at least some subjects, with direct toxicities on multiple other organ systems at standard dosing, often limiting their broader use. In fact, Amiodarone may represent ˜30% of the world's antiarrhythmic drug market, however nearly ⅓ of all patients cannot tolerate long-term use of the drug due to its extra-cardiac adverse reactions that affect nearly every organ in the body including the thyroid, lungs, liver, eyes, skin, central and peripheral nervous system among others. Direct targeting of antiarrhythmics such as Amiodarone to the CCS with the compositions and methods according to this disclosure may have the potential to improve treatment of life-threatening cardiac arrhythmias (e.g., junctional ectopic tachycardia) while at the same time mitigating their serious off-target effects. Finally, as the CCS is composed of multiple distinct components, each of which have different functions and are subject to different types of arrhythmias, a true precision approach to antiarrhythmic medication delivery would require the ability to target each subcomponent which can be accomplished with regents and compositions according to this disclosure.

In another aspect, this disclosure relates to a recombinant nucleic acid comprising the nucleic acid with SEQ ID NO. 2 which is encoding the light chain variable region (V_{L}) with SEQ ID NO: 1, SEQ ID NO. 4 which is encoding the light chain variable region (V_{L}) with SEQ ID NO: 3, SEQ ID NO. 6 which is encoding the heavy chain variable region (V_{H}) with SEQ ID NO: 5 and/or SEQ ID NO. 8 which is encoding the heavy chain variable region (V_{H}) with SEQ ID NO: 7, or a nucleic acid variant with at least 70% nucleic acid sequence identity with SEQ ID NO. 2, SEQ ID NO. 4, SEQ ID NO. 6 and/or SEQ ID NO. 8, said variant nucleic acids encoding functional variants of the light chain and heavy chain variable regions according to this disclosure.

Further aspects of this disclosure include recombinant DNA or RNA constructs, e.g., plasmids, cosmids, artificial chromosomes, which express one or more antibodies or antigen binding fragments, or one or more epitopes of this disclosure. Preferably, the recombinant construct may comprise a recombinant nucleic acid comprising the nucleic acid with SEQ ID NO. 2 which is encoding the light chain variable region (V_{L}) with SEQ ID NO: 1, SEQ ID NO. 4 which is encoding the light chain variable region (V_{L}) with SEQ ID NO: 3, SEQ ID NO. 6 which is encoding the heavy chain variable region (V_{H}) with SEQ ID NO: 5 and/or SEQ ID NO. 8 which is encoding the heavy chain variable region (V_{H}) with SEQ ID NO: 7, or a nucleic acid variant with at least 70% nucleic acid sequence identity, preferably at least 80%, more preferably at least 90% and most preferably at least 95%, with SEQ ID NO. 2, SEQ ID NO. 4, SEQ ID NO. 6 and/or SEQ ID NO. 8, said variant nucleic acids encoding functional variants of the light chain and heavy chain variable regions according to this disclosure.

Further aspects of this disclosure also include recombinant cells, e.g., bacterial cells, yeast cells or mammalian cells, which comprise one or more recombinant DNA or RNA constructs according to this disclosure and which express one or more antibodies or antigen binding fragments, or one or more epitopes/antigens described in this disclosure.

A further description will now be provided by the way of the following non-limiting examples.

### Example 1. Methods

Mice: Wild-type, CD1 mice were acquired from Jackson Laboratory (Sacramento, CA). Mice at indicated ages were used in accordance with the Institutional Animal Care and Use Committee of Stanford University. Both female and male mice were used for all experiment types described at a 1:1 ratio.

Human CCS Tissue: Human cardiac conduction system tissue samples (Infant, n=1, 11 months; Adult, n=1, 45 years) were acquired from autopsies at the Stanford University Department of Pathology and were appropriately de-identified. Tissues were fixed for 24 hours in 4% paraformaldehyde (Fisher, 50-980-487), washed in PBS for 10 minutes three times and incubated in 30% sucrose in PBS for 24 hours at 4° C. and then embedded in Tissue-Plus OCT (Fisher, 23-730-571) for cryosection. Immunostaining as detailed below.

Bioinformatics Analysis: All bioinformatics analyses were performed on our pre-existing single-cell RNA sequencing (scRNAseq) dataset of the developing mouse CCS as previously described,¹³ with additional details in Supplemental Methods. Significance is presented as an “adjusted p-value”, which is based on the Bonferroni correction using all features in the dataset. SurfaceGenie, a web-based application, was used to predict candidate surface marker from the pool of significantly enriched genes.¹⁴ All putative cell surface markers were then confirmed manually using UniProt.¹⁵

Optical Imaging Agents: Described antibody-dye conjugates consist of commercially acquired antibodies (1. anti-Cntn2 Goat Polyclonal antibody-AF4439; and 2. anti-NPTN Goat Polyclonal antibody-AF5360) that have been covalently conjugated to a benign, near-infrared (NIR) dye (IRDye800CW, Li-cor #929-70020) using company specifications. Control agents (IgG-800) consisted of non-specific IgG (Fisher, 50270683) conjugated to the same NIR dye. Unconjugated free dye was eliminated using Zeba™ Spin Desalting Columns (Pierce, #89891).

Human anti-CNTN2 Monoclonal Fab Creation: A phage display system was used (Promab Biotech, Inc) to screen for monoclonal Fab antibodies targeting the human CNTN2-His recombinant protein (Acro Biosystems, #CN2-H5226). See Supplemental Methods for details.

Monoclonal Fab In Vivo Targeting of the CCS: The monoclonal anti-CNTN2 Fab was conjugated to the same, aforementioned NIR dye (IRDye800CW, Li-cor, #929-70020) using company specifications. For conjugation to saporin, the Fab was first biotinylated using the fluoreporter mini-biotin-XX protein labeling kit (Invitrogen, #F6347) according to company specifications. The biotinylated-Fab (100 ug) was then added to streptavidin-conjugated Saporin (25 ug) (ATS, #IT-27) at room temperature prior to injection into wild-type CD1 mice. Control mice were injected with an equivalent amount of random human IgG conjugated to Saporin (ATS, #IT-27). Surface electrocardiograms were taken prior to injection and daily under inhaled sedation until euthanasia after 48 hours. Following euthanasia, the heart was then harvested, fixed in 4% paraformaldehyde for 24 hours prior to washing in PBS for 10 minutes three times. Hearts were then embedded in OCT, sectioned and immunostained as detailed below.

Delivery of Optical Imaging Agents, Surface ECG and Imaging: Adult, CD1 mice received systemic injections of either mNptn-800 or mCntn2-800 at indicated doses, by tail-vein injection under inhaled sedation. Controls consisted of mice injected with random IgG conjugated to the same NIR dye (IgG-800). Surface electrocardiograms were taken prior to injection and daily under inhaled sedation until euthanasia after 24, 48 or 72 hours as indicated. Following euthanasia, the heart, along with all other major organs were then harvested and imaged using closed-field (Pearl Impulse, LI-COR, Lincoln, NE) fluorescence imaging. Closed-field fluorescence images were analyzed with ImageStudio (LI-COR) by calculating mean fluorescence intensity (MFI) within a tailored region of interest (ROI). The ROI was hand drawn around the sinoatrial nodal (SAN) tissue to quantify conduction tissue MFI. To assess background MFI, an ROI was created on the left atrial appendage (LAA). The conduction-to-background MFI ratio (Signal to Background Ratio or “SBR”) was assessed for each mouse to evaluate the temporal effect on the fluorescence contrast produced by each agent.

Immunofluorescence: Immunofluorescence staining was carried out by following a previous protocol with minor modifications.¹⁶ Briefly, all tissue samples were washed in PBS prior to fixation overnight in 4% paraformaldehyde (Fisher, 50-980-487) at 4° C. Hearts were then washed in PBS for 15 min three times prior to incubation in 30% sucrose in PBS overnight at 4° C. and then embedded in Tissue-Plus OCT (Fisher, 23-730-571). Tissues were cut as cryosections of 10 μM thickness and stored at −80° C. The sections were dried for 1 hour prior to use, rehydrated in PBS, washed three times in PBST (PBS+0.1% Triton X100) and then blocked (PBST+0.5% Bovine serum albumin) for 1 hour at RT. Following this, the sections were incubated with primary antibodies diluted in blocking solution overnight at 4° C. in humid chambers. On the second day, after washing three times with PBST, the sections were incubated with secondary antibody for 2 hours at room temperature. After additional washing with PBS for 5 minutes three times, the sections were mounted with mounting media containing DAPI (Vector Laboratories, H-1200). All primary and secondary antibodies used are detailed in Supplemental Methods. Images were taken with Axioimager microscope at Neuroscience Microscope Service (NMS) facility at Stanford University. Negative controls for immunostaining included the use of primaries or secondary antibodies alone. A minimum of 4 biological (different hearts) and 4 technical (different slides/heart) replicates were used for each antibody staining.

RNAscope in situ hybridization: RNAscope® Multiplex Fluorescent v2 (Cat. #323100) was used per manufacturer suggested protocol. The following murine probes were used: Mm-Cpne5-C3-Cat No. 496711-C3, Mm-Hcn4-C2-421271-C2, Mm-Ntm-C1-Cat No. 489111, Mm-Pcdh17-C2-Cat No. 489901-C2, Mm-Slc22a1-C1-Cat No. 532931, Mm-Slit2-C1-Cat No. 449691, and Mm-Slitrk5-C1-Cat No. 451891. All images were taken with Axioimager microscope at Neuroscience Microscope Service (NMS) facility at Stanford University. A minimum of 3 biological (different hearts) and 4 technical (different slides/heart) replicates were used for each in situ hybridization.

iDISCO+: For detailed protocol, please see https://idisco.info/idisco-protocol/. Hearts acquired from mice systemically injected with mCntn2-800 were fixed and optically cleared per protocol. As the fluorescent probe mCntn2-800 was injected intravenously already prior to fixation, no primary or secondary antibodies were applied. Permeabilization was deferred given the lack of need for incubation with additional antibodies. At least one day after clearing, iDISCO+ samples were imaged on a light sheet microscope (Ultramicroscope II, LaVision Biotec). A minimum of 4 biological (hearts from separately injected mice) replicates were used for each optical clearing.

### Example 2. In Vivo Visualization of the Cardiac Conduction System Using mCntn2-800

To assess the feasibility of visualizing the CCS in real-time, we conjugated IRDye800CW, a near infrared (NIR) dye already in clinical use,¹⁷ to a commercially available polyclonal antibody directed against Cntn2, an extracellular marker previously shown to be expressed specifically within the CCS of mice and humans (FIG. 1A).^{18,19} We injected wild-type adult mice intravenously with a single dose of either mCntn2-800 (75 ug) or control IgG-800 (i.e. non-reactive IgG conjugated to IRDye800CW) and harvested the hearts and all other major organs after 72 hours and imaged them using a closed-field NIR imaging system (FIG. 1B). NIR signal was detected expectedly within the liver and kidneys similar to prior reports of metabolism and clearance of other NIR imaging agents.²⁰ Notably, mCntn2-800 signal was not detected within the brain tissue despite it being the only other major organ besides the CCS known to express Cntn2,²¹ consistent with an intact blood-brain barrier (FIG. 1C).

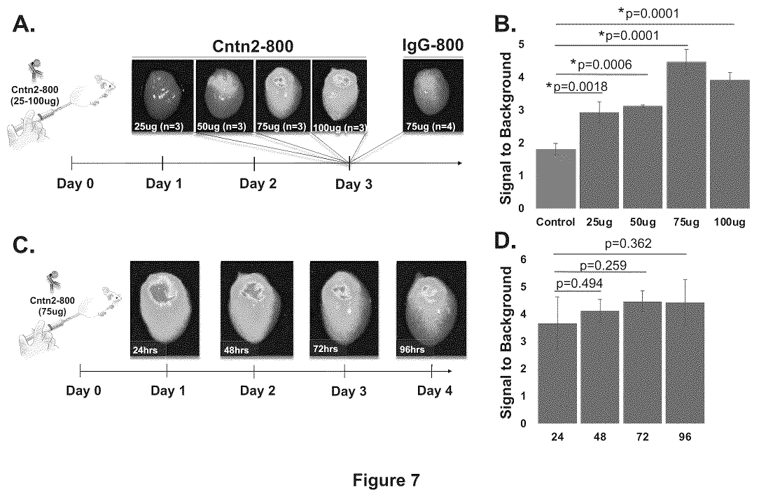

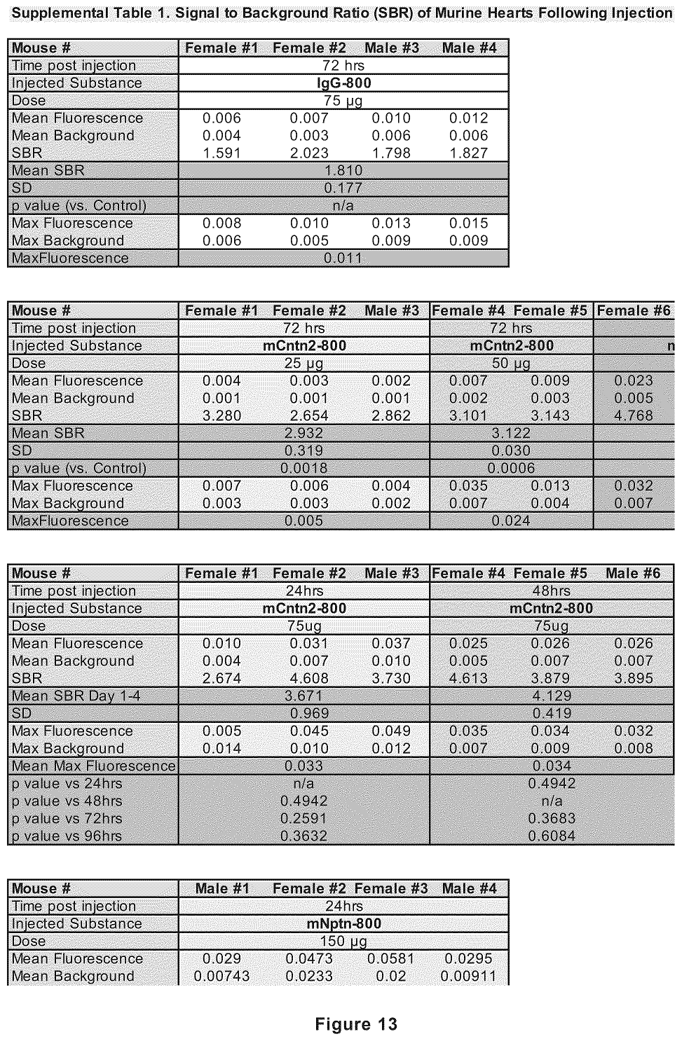

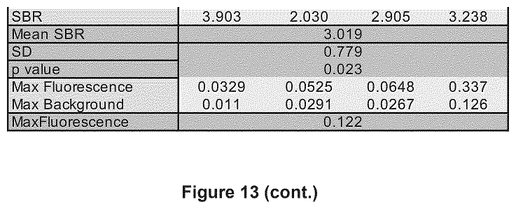

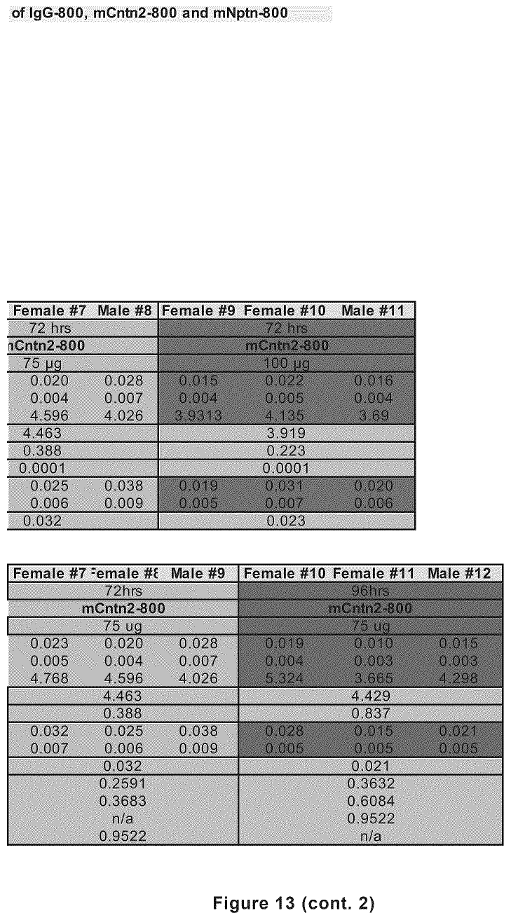

Within the heart, mCntn2-800 showed high intensity signal localized to the CCS (signal to background ratio, SBR: 4.463, SD: 0.388) as compared to mice injected with control IgG-800 (SBR: 1.810, SD: 0.177; p=0.0001) (FIG. 1D; FIG. 13 (Supplemental Table 1)). A dose finding study revealed that specific imaging signals were present at all doses given, with an optimal SBR at 75 ug (˜2 mg/kg) but with significant signal present even at the lowest dose of 25 ug (˜0.7 mg/kg) (SBR: 2.932, SD: 0.319; p=0.0018) (FIG. 7 (Supplemental FIG. 1); FIG. 13 (Supplemental Table 1)). A clearance study revealed that a single 75 ug dose of mCntn2-800 provides specific and intense signal even out to 4 days after the initial injection (FIG. 7 (Supplemental FIG. 1); FIG. 13 (Supplemental Table 1)).

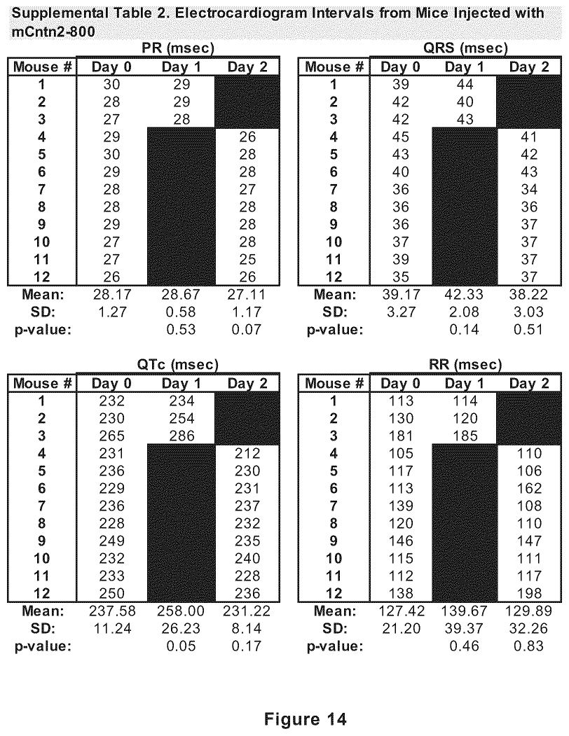

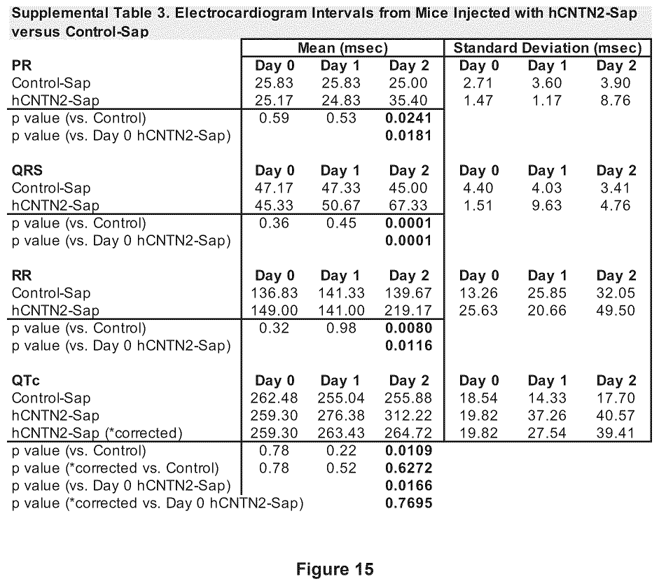

To address potential toxicity of mCntn2-800 on the CCS, a surface electrocardiogram (ECG) study was performed for two days after a single 75 ug injection of mCntn2-800. This study revealed no adverse effect of mCntn2-800 on normal CCS function as demonstrated by no difference in all surface ECG parameters when compared with the mice prior to injection (FIG. 1E; FIG. 14 (Supplemental Table 2)). Finally, we examined the specificity of mCntn2-800 to target the CCS and confirmed the absence of their binding to non-CCS cells by serial sections and co-immunostaining with established markers of the CCS (FIG. 1F-H).

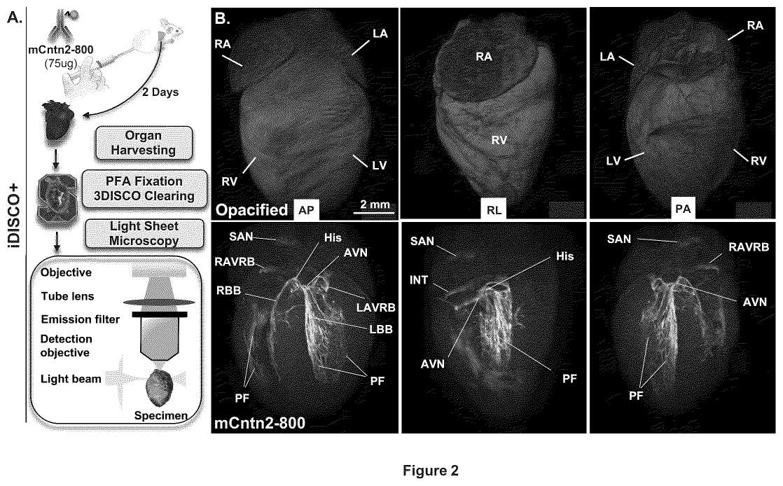

Given the challenges of visualizing the complex three-dimensional anatomy of the CCS, we leveraged whole-mount immunostaining and 3D imaging using tissue clearing (iDISCO+)²² and light sheet microscopy with volume rendering on intact, wild-type mouse hearts (FIG. 2A). Specifically, following a single 75 ug injection, whole hearts were harvested after 48 hours, fixed and cleared using iDISCO+. Consistent with 2D immunofluorescence sections, the cleared hearts showed exquisite specificity and high resolution of mCntn2-800 throughout the entire CCS (FIG. 2B).

### Example 3. Generation and Characterization of a Fully Human Anti-CNTN2 Fab Antibody that Targets the CCS In Vivo

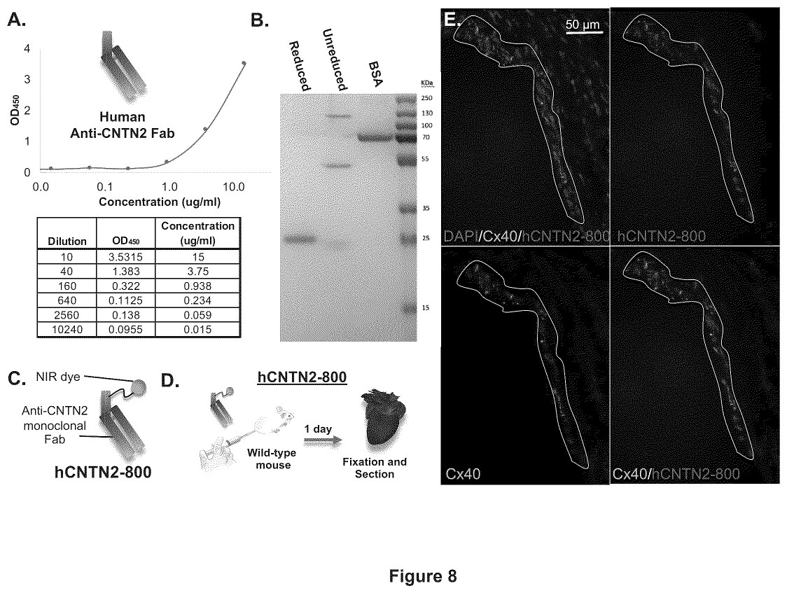

To facilitate clinical translation and with support from a commercial vendor, we employed a phage display strategy to screen for a fully human monoclonal antibody Fab that binds with high affinity to the human recombinant CNTN2. From initial hits, we validated our lead Fab to bind human recombinant CNTN2 in vitro (FIG. 8A-B (Supplemental FIG. 2A-B)) and subsequently generated a Fab-IRDye800CW conjugate (hCNTN2-800) by NHS ester chemistry (FIG. 8C (Supplemental FIG. 2C)). hCNTN2-800 was then introduced into wild-type mice by a single tail-vein injection. After 1 day, their hearts were harvested, fixed and sectioned for immunofluorescence staining (FIG. 8D (Supplemental FIG. 2D)). Immunofluorescence co-staining with connexin 40, a well-known marker of Purkinje fiber cells, demonstrates that hCNTN2-800 can successfully target the CCS in vivo (FIG. 8E (Supplemental FIG. 2E)).

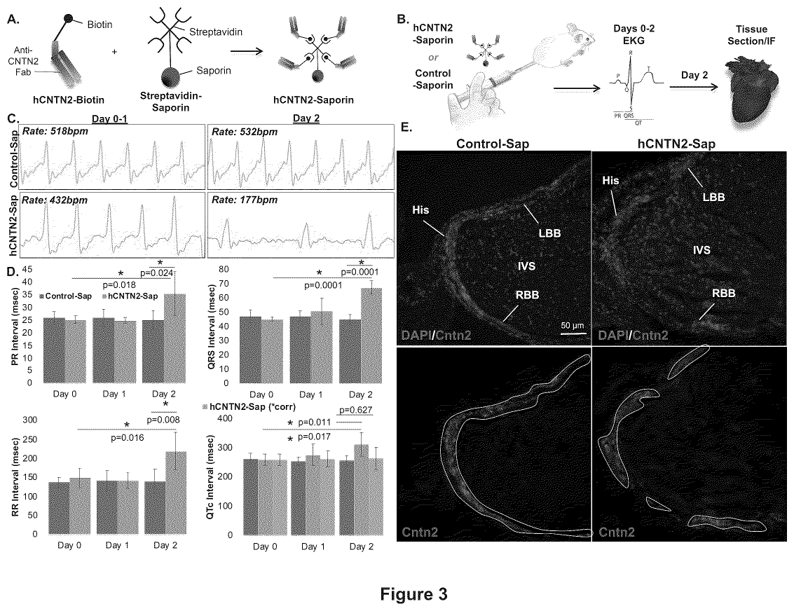

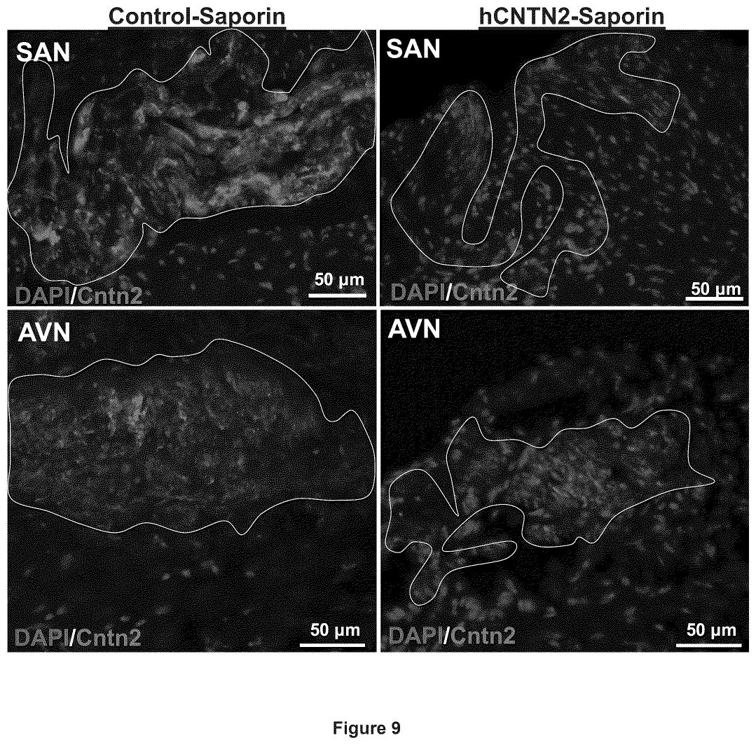

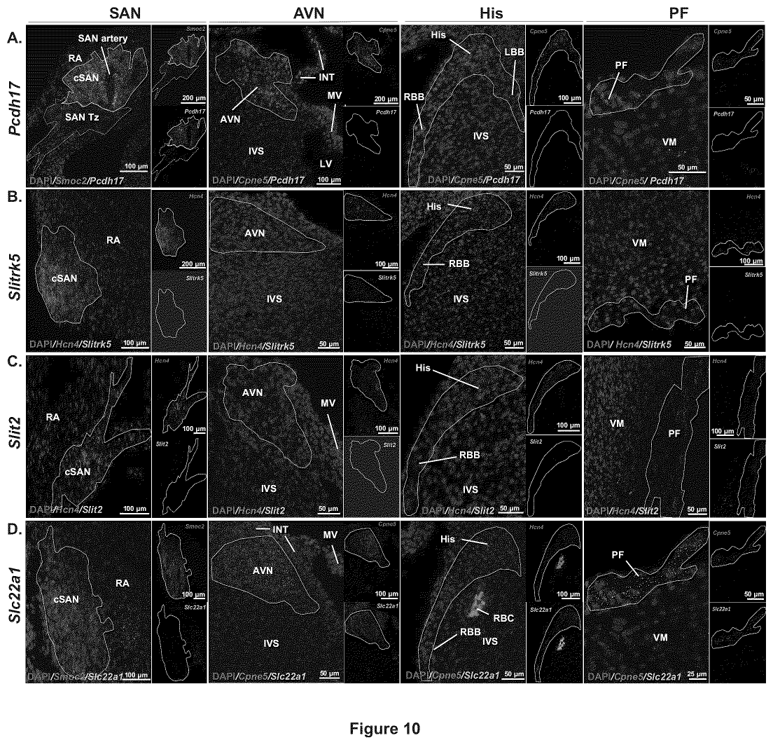

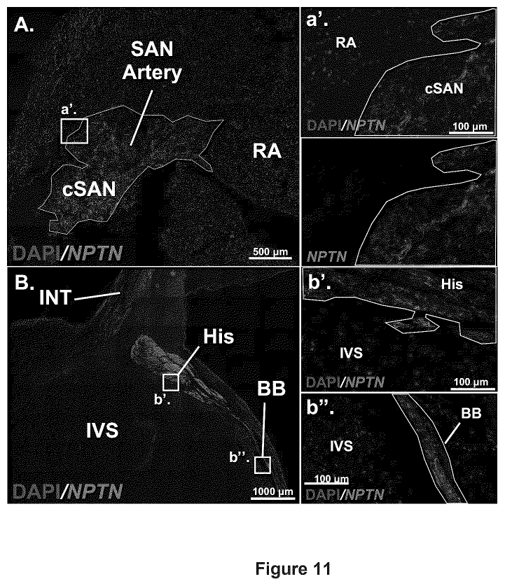

Beyond their use in real-time in vivo imaging of the CCS, the availability of a CCS targeting antibody may also facilitate the treatment of arrhythmias by precision delivery of drugs or alternative cargos capable of modulating CCS cell function. To examine the ability of our monoclonal Fab to target and functionally perturb the CCS, we biotinylated the hCNTN2 Fab and conjugated it to streptavidin bound to Saporin, a cellular toxin that is incapable of receptor-mediated internalization through cell surface membranes. The incubation of these two products resulted in “hCNTN2-Sap” (FIG. 3A). We then injected wild-type mice with a single tail-vein injection of either hCNTN2-Sap (100 ug, n=6) or Control-Sap (100 ug of non-reactive human IgG conjugated to Saporin, n=6) and harvested hearts 2 days later (FIG. 3B). These mice received baseline (Day 0) and daily ECGs following injection to assess for conduction system disruption. At Day 2 post-injection, mice injected with hCNTN2-Sap consistently showed markedly abnormal cardiac rhythm including prolonged RR (219 ms vs. 140 ms in Controls; p=0.008), PR (35 milliseconds [ms] vs. 25 ms in Controls; p=0.024), and QRS (67 ms vs. 45 ms in Controls; p=0.0001) intervals (FIG. 3C-D), consistent with abnormal function within the SA node, AV node, and Bundle branches/Purkinje fibers, respectively. While QTc intervals, reflective of ventricular myocardium repolarization, were also prolonged (312 ms vs. 256 ms in Controls; p=0.011), when corrected for the widened QRS intervals, no significant difference was noted (265 ms vs. 256 ms in Controls; p=0.627), again consistent with conduction cell-specific targeting within the heart. We then confirmed these ECG findings with immunohistochemical analyses of the CCS structure and found that the CCS of hCNTN2-Sap-injected mice showed wide-spread loss of cardiac conduction cells in all major CCS components consistent with their targeted cell-death (FIG. 3E; FIG. 9 (Supplemental FIG. 3)).

### Example 4. Gene Expression Analysis of Murine CCS Single-Cell RNA-Sequencing Dataset Reveals Novel Cell Surface Genes Enriched within the CCS

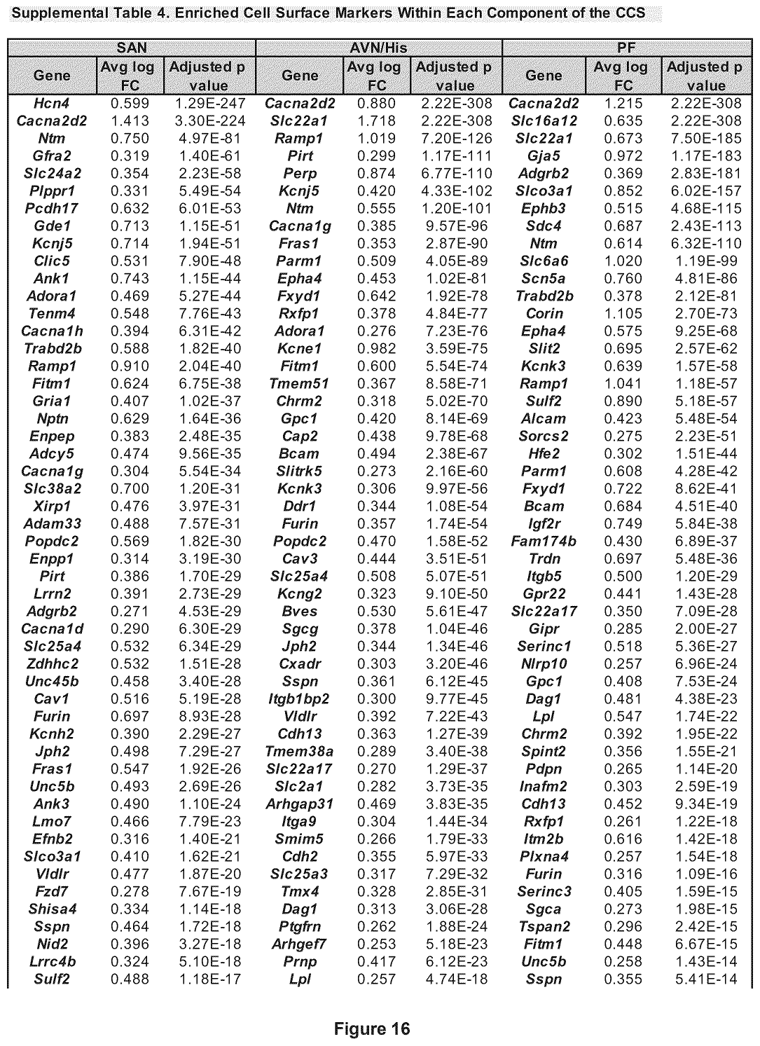

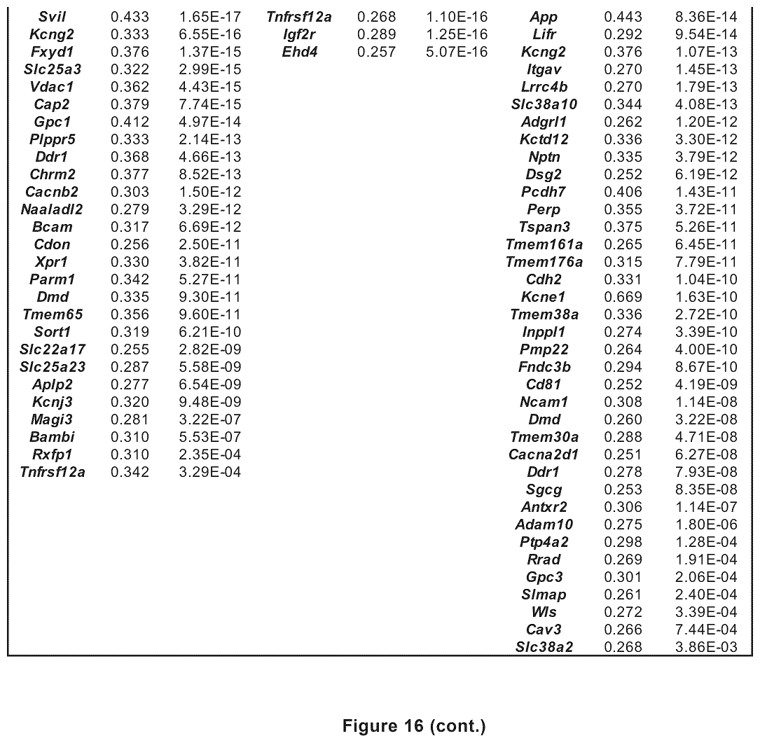

Given our successes thus far with antibody targeting of Cntn2 for in vivo imaging and phenotypic modulation of CCS cells, we next sought to identify additional highly specific cell surface markers within the CCS for substructure-specific targeting. To do this, we leveraged our previously validated single-cell RNA-sequencing (scRNA-seq) dataset of the entire developing murine CCS.¹³ Specifically, we screened for all significantly enriched genes within single cells from each major subcomponent of the CCS, including the sinoatrial node (SAN), atrioventricular node (AVN), His bundle (His) and Purkinje fiber (PF) cells as compared to all other cells within the heart. We prioritized our genes of interest by: 1. Log fold enrichment within the CCS as compared to other cell types; 2. Significance (“adjusted p-value” based on the Bonferroni correction); and 3. Their inclusion using the recently published Surface Genie algorithm,¹⁴ a web-based application for high-throughput candidate surface marker prioritization. All putative cell surface genes were then confirmed by manual assessment of their subcellular localization using UniProt Knowledgebase.¹⁵ These analyses resulted in the discovery of a number of CCS-enriched cell surface genes, both known (eg. SAN-Hcn4: 0.599 avg log FC, adjusted p value=1.29×10⁻²⁴⁷; AVN/His-Cav3: 0.444 avg log FC, adjusted p value=3.51×10⁻⁵¹; PF-Gja5: 0.972 avg log FC, adjusted p value=1.17×10⁻¹⁸³)^{23,24} and novel within the CCS (FIG. 16 (Supplemental Table 4)).

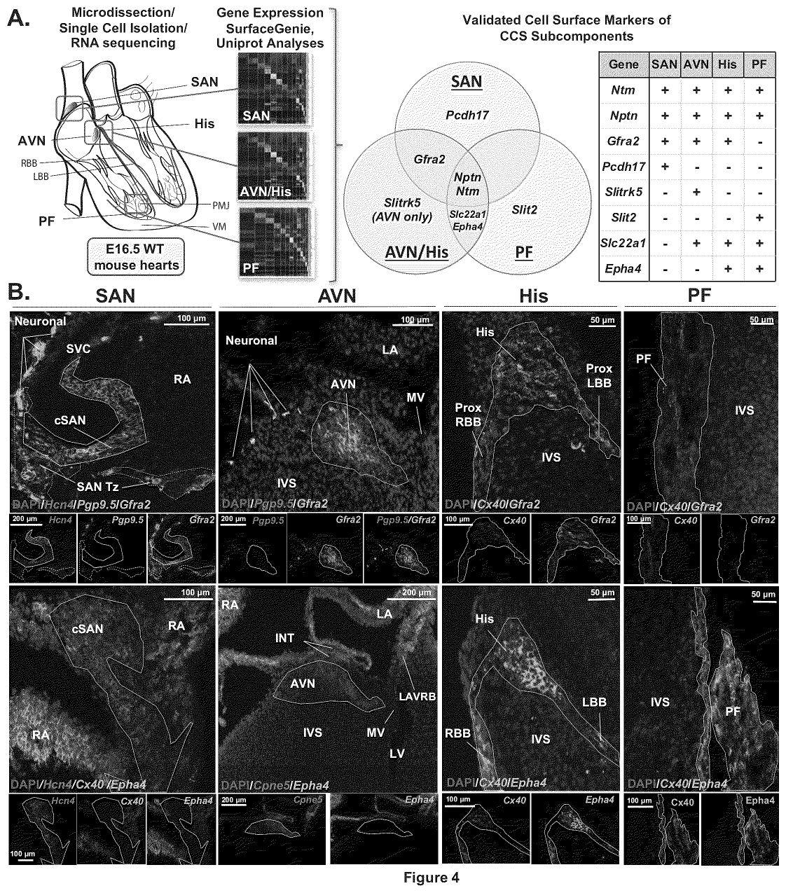

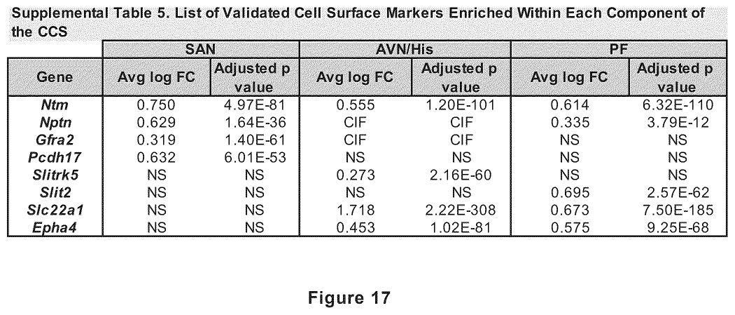

From this list of novel CCS cell surface genes, we identified ones enriched within each distinct CCS component including the SAN (Pcdh17: 0.632 avg log FC, adjusted p value=6.01×10⁻⁵³), AVN (Slitrk5: 0.273 avg log FC, adjusted p value=2.16×10⁻⁶⁰), and PF cells (Slit2: 0.695 avg log FC, adjusted p value=2.57×10⁻⁶²) (FIG. 4A; FIG. 17 (Supplemental Table 5)). Additional cell surface genes were found to be enriched in distinct combinations of subcomponents including the nodal tissue (SAN/AVN/His-Gfra2), ventricular conduction system (AVN/His/PF-Slc22a1), distal fast-conduction CCS (His/PF-Epha4), as well as throughout the entire CCS (Neurotrimin [Ntm] and Neuroplastin [Nptn]) (FIG. 4A; FIG. 17 (Supplemental Table 5)). While Nptn was not initially found to be significantly enriched within AVN/His cells when compared to all other cell types, it was indeed significantly enriched when compared specifically to all other cardiomyocyte cell clusters (AVN/His-Nptn: 0.303 avg log FC, adjusted p value=4.69×10⁻²²) (data not shown).

### Example 5. Validation of Novel Cell Surface Markers Within Distinct Components of the Murine CCS

Consistent with our bioinformatics approach, one of the most enriched cell surface markers uncovered in our analyses was Neurotrimin (Ntm), encoding a member of the IgLON (LAMP, OBCAM, Ntm) family of immunoglobulin (Ig) domain-containing glycosylphosphatidylinositol (GPI)-anchored cell adhesion molecules, that we have previously validated to be specifically expressed throughout the entire CCS.¹³ In order to validate the other seven cell surface candidates, we next employed high resolution fluorescence in situ hybridization (RNAscope) or immunostaining analyses of wild-type murine heart sections. The cell type expression pattern of each gene candidate predicted from our scRNAseq expression data (FIG. 4A) were fully recapitulated by tissue staining in wild-type mouse hearts with high specificity, including Pedh 17 (SAN), Slitrk5 (AVN), Slit2 (PF), Gfra2 (SAN/AVN/His), Slc22a1 (AVN/His/BB/PF), and Epha4 (His/BB/PF) (FIG. 4B; FIG. 10 (Supplemental FIG. 4)).

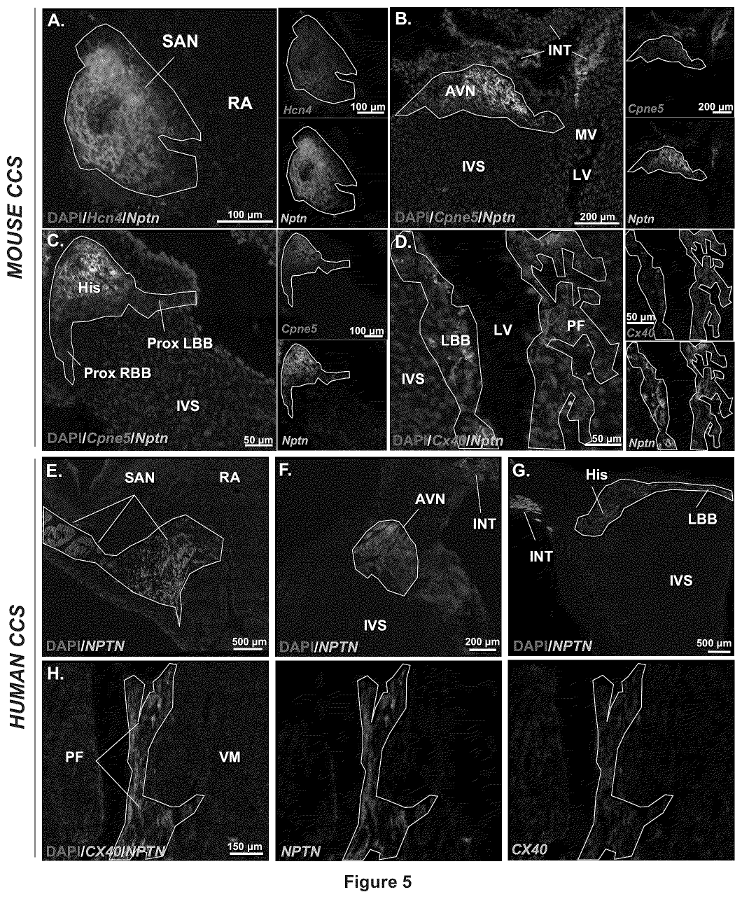

To validate the in vivo targeting specificity of a novel CCS marker identified from our scRNAseq dataset, we focused on the expression of Neuroplastin (Nptn), a type I transmembrane protein belonging to the Ig superfamily expressed within the central nervous system²⁵ in the murine and human heart (FIG. 5). Nptn was found histologically to be enriched throughout the entire CCS in both adult mice (FIG. 5A-D) and humans in early postnatal (FIG. 5E-H—11 month old) and adult (FIG. 11 (Supplemental FIG. 5)—45 years old) heart tissues.

### Example 6. In Vivo Labeling and Visualization of the Murine CCS by Nptn Targeting

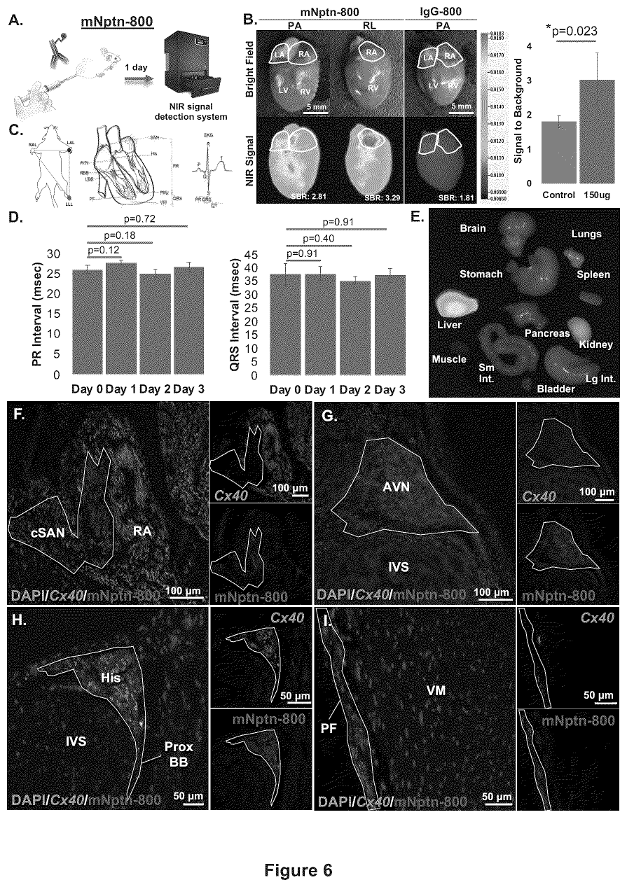

To confirm the ability of Nptn antibodies to specifically target the CCS, we engineered a “mNptn-800” by conjugating a commercial polyclonal anti-mouse Nptn antibody to IRDye800CW.¹⁷ Following a single intravenous tail vein injection of mNptn-800 or control IgG-800 (non-reactive IgG conjugated to IRDye800CW) in wild-type adult mice, we isolated whole hearts after 24 hours and imaged using a closed NIR camera system (FIG. 6A). We found that while IgG-800 showed no specific signal within the hearts of injected mice (SBR: 1.81, SD: 0.177), a strong and CCS-specific signal was detected in mNptn-800-injected hearts (SBR: 3.02, SD: 0.779, p=0.023) (FIG. 6B; FIG. 13 (Supplemental Table 1)).

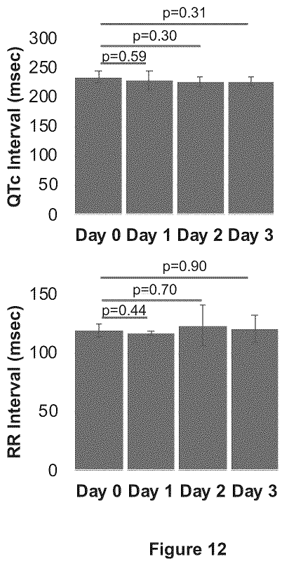

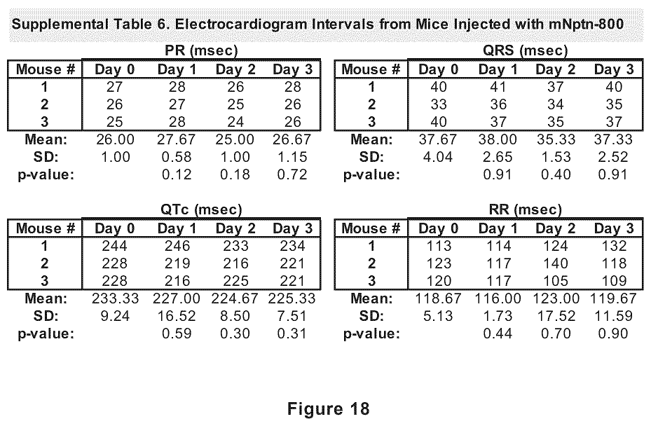

To assess for potential toxicities of mNptn-800 on CCS function, all mice received pre-injection baseline (Day 0) and daily ECGs for up to 3 days following injection (FIG. 6C-D; FIG. 12 (Supplemental FIG. 6); FIG. 18 (Supplemental Table 6)).

Reassuringly, all measured ECG intervals (including PR, QRS, QTc and RR) remained unchanged following systemic injection of mNptn-800 compared with IgG-800, consistent with a lack of toxicity to the CCS by mNptn-800.

We next assessed the biodistribution of mNptn-800 in other organs using closed-field NIR imaging. Following systemic delivery of the optical imaging agent, organs were freshly harvested and signal was detected expectedly within the liver and kidneys, similar to mCntn2-800 and clearance of other optical imaging agents in these organs (FIG. 6E).²⁰ No signal was detected within the brain tissue despite the known expression of Nptn within the central nervous system,²⁵ consistent with the exclusion of mNptn-800 by an intact blood-brain barrier. Finally, to assess signal specificity of mNptn-800 at the cellular level, serial heart sections of systemically injected mice were co-stained with known protein markers of the CCS such as connexin 40. mNptn-800 signal was localized specifically to all components of the CCS but not to surrounding heart muscle tissue (FIG. 6F-I).

### Example 7. Supplemental Methods

Both female and male mice were used for all experiment types described at a 1:1 ratio. For systemic injection of novel optical imaging tools, adult male and female mice were used at the age of 4-6 weeks. For immunofluorescence or FISH analyses, mice were used at indicated ages.

Human CCS Tissue: Human cardiac conduction system tissue samples (Infant, n=1, 11 months; Adult, n=1, 45 years) with post-mortem intervals (PMI) less than 24 hours were acquired from autopsies at the Stanford University Department of Pathology and were appropriately de-identified. Hearts were fixed for 24 hours in 4% paraformaldehyde (Fisher, 50-980-487) and washed in PBS for 10 minutes three times (Thermofisher, 10010023). Conduction components including the SAN region (SVC-RA junction), AVN region (triangle of Koch) and PF region (free wall and IVS) were manually dissected and incubated in 30% sucrose in PBS for 24 hours at 4° C. and then embedded in Tissue-Plus OCT (Fisher, 23-730-571). Tissue sections were cut as cryosections of 12 μM thickness and stored at −80° C. Immunostaining as detailed below.

Bioinformatics Analysis: All bioinformatics analyses were performed on our pre-existing single-cell RNA sequencing (scRNAseq) dataset of the developing mouse CCS.18 The Droplet platform data was de-multiplexed and mapped to mouse genome MM10 using CellRanger from 10× Genomics with default parameters. Cell filter, data normalization, and unsupervised analysis were carried out in Seurat version 2 per their recommended steps.19,20 Significance is presented as an “adjusted p-value”, which is based on the Bonferroni correction using all features in the dataset. Briefly, the cells were filtered by their gene number and UMI number. The threshold we used for gene number is 500 to 60,000, and UMI number is 1,000 to 5 million. Next, we used the LogNormalize function to normalize gene expression in each cell. Specifically, we calculated the expression value of genes by following this formula: log {(each gene expression level/total gene expression value)*10,000}. Average log fold change (avg_log FC) described in all data provided represents the log fold-change of the average expression between the two groups. To remove the unwanted sources of variations, we scaled the data with the “vars.to.regress” parameter based on the number of UMIs, percentage of ribosome genes, and Rn45s expression value. Furthermore, we found all the variable genes and used them to perform principal component analysis (PCA). Within all the PCs, we used the top 10 PCs to do clustering and tSNE analysis. In the tSNE analysis, we set the seed.use as 10 and perplexity as 30. Finally, we used the FindAllMarker function to identify the genes differentially expressing in the cell clusters. To be detected, the genes have to express in at least 25% of cells in one of the two comparing clusters and the differential expression level also should be higher than 25%. SurfaceGenie, a webbased application, was used to predict candidate surface marker from the pool of significantly enriched genes.21 All putative cell surface markers were then confirmed manually using UniProt.22

Description of Optical Imaging Agents: The generated optical imaging agents consist of commercially acquired antibodies (1. anti-Cntn2 Goat Polyclonal antibody-AF4439; and 2. anti-NPTN Goat Polyclonal antibody-AF5360) that have been covalently conjugated to a benign, near-infrared (NIR) dye (IRDye800CW, Li-cor #929-70020) using company specifications. IRDye800CW is a NIR imaging probe with broad absorption (778 nm) and emission (794 nm) peaks that is nontoxic to rodents23 and currently used in human clinical imaging trials.7 Control agents (IgG-800) consisted of non-specific IgG (Fisher, 50270683). In brief, 100 μg of polyclonal antibody (diluted in 100 uL PBS) was pH adjusted to a pH of 8.5 by adding 10 μL of 1.0 M potassium phosphate buffer (K2HPO4; pH 9). Next, one vial of IRDye800CW NHS ester was dissolved with 25 μL of ddH20. Dye and antibody were immediately mixed to achieve a dye/protein ratio of 1.5-2:1 and kept at RT in the dark for 2 hours. After 2 hours of incubation, the antibody-IRDye800CW conjugation mixture was put onto PBS-equilibrated Zeba™ Spin Desalting Columns (pH 7.4) (Pierce, #89891) and centrifuged at 1,500×g for 2 min to separate conjugate from free dye. Conjugated agents were then stored at 4° C., in the dark.

Human anti-CNTN2 Monoclonal Fab Creation: A phage display system was used (Promab Biotech, Inc) to screen for monoclonal Fab antibodies targeting the human CNTN2-His recombinant protein (Acro Biosystems, #CN2-H5226). This recombinant human Contactin-2, His Tag (CN2-H5226) contains amino acids Ser 31 through Asn 1012 of SEQ ID NO: 9. This recombinant human Contactin-2 carries a polyhistidine tag at the C-terminus. The protein has a calculated MW of 108.3 kDa. The protein migrates as 116-130 kDa under reducing (R) condition (SDS-PAGE) due to glycosylation.

In the 1st round of selection, the target antigen was biotinylated and coated on Streptavidin Dynal beads. The phage library was incubated with the target protein at 100 nM in 1 ml PBS containing casein as the blocking agents. Following 1.5 hours binding, the beads were washed 7 times and the bound phage were eluted by 100 mM triethylamine and amplified by infecting TG1 cells and rescued by M13KO7 helper phage. About 8e5 clones were recovered in this round. In the 2nd round of selection, the procedures followed those in the 1 st round selection except the antigen concentration was reduced to 10 nM and 1e11 phage was used in 0.5 ml PBS/casein volume, and the wash was increased to 15 times. Upon elution, the phage was used to infect HB2151 cells and 1.2e6 clones were recovered. Fab expression: Single colonies were inoculated in 96-wells in 2×YT medium containing ampicillin and 2% glucose and grown at 37 degree for 5 hours. The culture was diluted 50-fold in 2×YT medium containing ampicillin and grown for 2 hours. After adding IPTG at a final 1 mM, the cultures were grown at 30 degree overnight to induce soluble Fab expression.

ELISA screening: target protein Contactin2-binding Fab clones were identified by ELISA. 96-well plates were coated with target protein at 2 μg/ml overnight.

Following blocking, crude supernatants were added to the wells. Following the wash, anti-human Fab-HRP was added. Positive clones were identified using TMB as the substrate. A total of 20×96 clones were screened resulting in 15 positive hits. 2nd ELISA to confirm the positive hits: ELISA was repeated for the 15 clones using target protein and negative control protein-coated wells, in duplicate. All 15 clones were sequenced by Sanger's method and 12 were found to be unique sequences. Two clones were ultimately selected for in vivo experimentation based on their higher yield following culture and purification and binding affinity.

Monoclonal Fab In Vivo Targeting of the CCS: The monoclonal anti-CNTN2 Fab was conjugated to the same, aforementioned nearinfrared (NIR) dye (IRDye800CW, Li-cor, #929-70020) using company specifications. For conjugation to saporin, the Fab was first biotinylated using the fluoreporter minibiotin-XX protein labeling kit (Invitrogen, #F6347) according to company specifications. The biotinylated-Fab (100 ug) was then added to streptavidin-conjugated Saporin (25 ug) (ATS, #IT-27) at room temperature prior to injection into each wild-type CD1 mouse. Six biological replicates were performed and compared to six control CD1 mice injected with an equivalent amount of random human IgG conjugated to Saporin (ATS, #IT-27). Surface electrocardiograms were taken prior to injection and daily under inhaled sedation until euthanasia after 48 hours. Following euthanasia, the heart was then harvested, fixed in 4% paraformaldehyde for 24 hours prior to washing three times 10 minutes in 1×PBS. Hearts were then embedded in OCT, sectioned and stained as detailed in immunofluorescence methods.

Delivery of Optical Imaging Agents, Surface ECG and Imaging: For systemic application, adult, CD1 mice were administered of either mNptn-800 (150 ug) or mCntn2-800 (75 ug), diluted in 100 ul of sterile PBS (Thermofisher, 10010023), by tail-vein injection under inhaled sedation (isofluorane 3.5%). Controls consisted of mice injected with IgG-800 (75 ug). Surface electrocardiograms were taken prior to injection and daily under inhaled sedation until euthanasia after 24, 48 or 72 hours as indicated. Following euthanasia, the heart, along with all other major organs were then harvested and imaged using closed-field (Pearl Impulse, LI-COR, Lincoln, NE) fluorescence imaging (FLI). Subsequently, each heart was processed for immunofluorescence or iDISCO+ as detailed below.

Closed-field fluorescence images were analyzed with ImageStudio (LI-COR) by calculating mean fluorescence intensity (MFI) within a tailored region of interest (ROI). The ROI was hand drawn around the sinoatrial nodal (SAN) tissue to quantify conduction tissue MFI. To assess background MFI, an ROI was created on the left atrial appendage (LAA). The conduction-to-background MFI ratio (Signal to Background Ratio or “SBR”) was assessed for each mouse to evaluate the temporal effect on the fluorescence contrast produced by each agent.

Immunofluorescence: Immunofluorescence staining was carried out by following a previous protocol with minor modifications.24 Briefly, all tissue samples (including wildtype CD1 mouse hearts or human tissue sections from indicated gestational ages as well as hearts from postnatal mice previously injected with optical imaging agents) were isolated by dissection, washed in PBS prior to fixation overnight in 4% paraformaldehyde (Fisher, 50-980-487) at 4° C. Hearts were then washed in PBS for 15 min three times prior to incubation in 30% sucrose in PBS overnight at 4° C. and then embedded in Tissue-Plus OCT (Fisher, 23-730-571). Tissues were cut as cryosections of 10 μM thickness and stored at −80° C. The sections were dried for 1 hour prior to use, rehydrated in PBS, washed three times in PBST (PBS+0.1% Triton X100) and then blocked (PBST+0.5% Bovine serum albumin) for 1 hour at room temperature. Following this, the sections were incubated with primary antibodies diluted in blocking solution overnight at 4° C. in humid chambers. Primary antibodies used included: Antimouse Connexin 40 Rabbit Polyclonal (Alpha Diagnostics, Cx40-A) at 1:100 dilution; Anti-mouse/human/rat Cntn2/TAG1 Goat Polyclonal (R&D, AF4439) at 1:100 dilution; Anti-mouse EphA4 Goat Polyclonal (R&D, AF641) at 1:100 dilution; Anti-mouse/human GFRA2 Goat Polyclonal (R&D, AF429) at 1:100 dilution; Anti-mouse Hen4 Rat Monoclonal [SHG 1E5] (Abcam, ab32675) at 1:75 dilution; Anti-mouse NPTN55 Sheep Polyclonal (R&D, AF7818) at 1:200 dilution; Anti-mouse/human NPTN65 Goat Polyclonal (R&D, AF5360) at 1:100 dilution. On the second day, after washing three times with PBST, the sections were incubated with secondary antibody for 2 hours at room temperature. The following secondaries were used at a 1:500 dilution: Donkey anti-goat IgG Alexa Fluor 555 (Invitrogen, A-21432), Chicken anti-Rabbit IgG Alexa Fluor 488 (Invitrogen, A-21441), Donkey anti-Rabbit IgG Alexa Fluor 647 (Invitrogen, A-31573) and Chicken anti-Rat IgG AlexaFluor488 (Invitrogen A-21470). After additional washing with PBS for 5 minutes three times, the sections were mounted with mounting media containing DAPI (Vector Laboratories, H-1200). All images were taken with Axioimager microscope at Neuroscience Microscope Service (NMS) facility at Stanford University. Negative controls for immunostaining included the use of primaries or secondary antibodies alone. A minimum of 4 biological (different hearts) and 4 technical (different slides/heart) replicates were used for each antibody staining.

RNAscope in situ hybridization: RNAscope® Multiplex Fluorescent v2 (Cat. #323100) was used per manufacturer suggested protocol. The following murine probes were used: Mm-Cpne5-C3-Cat No. 496711-C3, Mm-Hen4-C2-421271-C2, Mm-Ntm-C1-Cat No. 489111, Mm-Pcdh17-C2-Cat No. 489901-C2, Mm-Slc22a1-C1-Cat No. 532931, Mm-Slit2-C1-Cat No. 449691, and Mm-Slitrk5-C1-Cat No. 451891. All images were taken with Axioimager microscope at Neuroscience Microscope Service (NMS) facility at Stanford University. A minimum of 3 biological (different hearts) and 4 technical (different slides/heart) replicates were used for each in situ hybridization. iDISCO+: For detailed protocol, please see https://idisco.info/idisco-protocol/. Hearts acquired from mice systemically injected with mCntn2-800 were fixed and optically cleared per protocol. As the fluorescent probe mCntn2-800 was injected intravenously already prior to fixation, no primary or secondary antibodies were applied. Additionally, the permeabilization step was deferred given the lack of need for incubation with additional exogenous antibodies. At least one day after clearing, iDISCO+ samples were imaged on a light sheet microscope (Ultramicroscope II, LaVision Biotec) equipped with a sCMOS camera (Andor Neo) and a 2×/0.5 NA objective lens (MVPLAPO 2×) equipped with a 6 mm working distance dipping cap. Version v285 of the Imspector Microscope controller software was used. We imaged using the 800-nm laser. The samples were scanned with a step-size of 3 μm using the continuous lightsheet scanning method. A minimum of 4 biological (hearts from separately injected mice) replicates were used for each optical clearing.

### Example 8. Discussion

Iatrogenic damage to the CCS, which surrounds many key interventional targets (e.g. heart valves) during cardiac procedures, can result in decreased heart function and a host of irreversible, life-threatening arrhythmias (e.g. heart block), often requiring permanent pacemaker device placement.²⁶ To address this unmet medical need, we generated a systemically injected, targeted molecular imaging tool (“mCntn2-800”), that allows for the real-time visualization of the CCS during invasive procedures with high sensitivity, specificity and resolution. mCntn2-800 represents, to our knowledge, the first ever method for the in vivo targeting of any cardiac substructure. Further, with the goal of translating our basic findings towards a viable prototype for human use, we generated a fully human monoclonal Fab, directed against human CNTN2 protein, that similarly targets the CCS with high specificity and is able to target CCS cells with other cargo that modulate CCS cell biology. Finally, in performing differential gene expression analyses of the entire murine CCS at single-cell resolution, we uncovered and validated a suite of additional cell surface markers that can be used to molecularly target the distinct subcomponents of the CCS.

Current standard of care in cardiac invasive procedures remains the use of crude anatomical landmarks to approximate the location of the CCS, otherwise indistinguishable from the surrounding heart muscle tissue. Over the past several decades attempts have been made towards the real-time detection of the CCS during intracardiac surgeries with the goal of preventing iatrogenic surgical damage. Earlier efforts dating back to the 1960s included measuring the impedance or electrocardiograms through the direct placement of electrodes on the heart tissue.²⁷ These approaches, however, have been hampered both by inadequate resolution, variable detection based on heterogeneity in CCS tissue depth and/or the need for ongoing electrical activity (i.e. not viable for surgeries requiring cardioplegia). More recent efforts in the past several years have employed fiberoptic confocal microscopy (FCM) coupled with a passive fluorescent dye applied directly to the tissue surface.²⁸ Using a hand-held FCM probe, the tissue surface can be evaluated for differences in tissue microstructure suggestive of conduction versus working myocardium.

Our antibody-based optical imaging approach provides several advantages including: 1) high specificity and spatial resolution; 2) lack of additional technical expertise needed to visualize the CCS; and 3) real-time visualization without disruption of the surgical field or workflow. Further, numerous features inherent to our antibody-based diagnostic tools help to promote viable clinical translation including: 1. Extensive research into the use of antibody-based therapeutics and diagnostics in humans, resulting in well-established pharmacokinetics and safety profiles;^{9.29} 2. Use of a near-infrared dye (NIR) suitable for human use and with deep signal penetration (up to ˜1 cm below the tissue surface),^{10,11} sufficient for even the AV node, the deepest of conduction structures;^{30.31} and 3. Pre-existing FDA-approved high-resolution intraoperative NIR signal detection devices such as SPY Elite (Stryker, USA). Integration of optical imaging diagnostic tools into the surgical management of cardiothoracic surgery has the potential to dramatically improve adverse outcomes in both pediatric and adult cardiac surgeries. Specifically, we envision that high-resolution, real-time CCS detection through direct, intraoperative visualization by surgeons will minimize the risk of iatrogenic damage, thereby reducing hospital costs and length of stay as well as the need for lifelong pacemaker dependency and overall morbidity and mortality associated with cardiac surgeries.

Importantly, our approach additionally lends itself to the broader potential of molecular targeting of the CCS in vivo, including systemic delivery of other forms of cargos such as contrast agents or therapeutics. Use of advanced imaging, including cardiac MRI and CT have become a critical aspect of preprocedural planning in invasive cardiac interventions.³² However, despite significant advances in these imaging modalities, they still cannot visualize the CCS within the heart, limiting their potential use. The lack of CCS visibility coupled with the large amount of anatomical variation, in particular in children with congenital heart disease (e.g. congenitally corrected transposition of the great arteries)³³ results in persistently elevated conduction-related complication rates in cardiac procedures.⁴ Antibody-contrast conjugates (ACCs), through the conjugation of our lead Fab with, for instance, either superparamagnetic iron oxide nanoparticles (SPION) (for MRI) or gold (Au) nanoparticles (for CT), have the potential to revolutionize cardiac imaging by unveiling, for the first time ever, the previously evasive location of the CCS of any patient using standard pre-procedural imaging.

Beyond the surgical suite, damage to the CCS is also a significant risk during invasive catheter-based procedures such as cardiac electrophysiology ablations, for which there are ˜40,000 adult cases per year in the US alone.³⁴ Pre-procedural mapping of the CCS using advanced imaging has the potential to revolutionize the field of electrophysiology by: 1) Improving ablation outcomes through direct visualization of target structures (e.g. slow pathway conduction fibers that serve as the nidus of atrioventricular nodal re-entrant tachycardia); 2) Minimizing the risk of iatrogenic damage of critical CCS components (e.g. detailing the proximity of pathways targeted for ablation in relation to other critical CCS structures); and 3) Helping to guide pacemaker device placement (e.g. His bundle lead placement).

Beyond dyes and contrast agents, our antibody-based delivery method also has therapeutic potential through the targeting of alternative cargos. Here we demonstrate CCS-specific targeted ablation following a single intravenous injection of our antibody-saporin conjugate, Currently, targeted ablation of the conduction system for rate control in refractory atrial fibrillation is highly effective however still necessitates an invasive electrophysiology ablation procedure.³⁵ These proof-of-principle experiments also highlight the potential for targeting other forms of cargo for precision therapy, namely antiarrhythmic drugs. Two of the most effective intravenous antiarrhythmics available are Amiodarone and Procainamide. However both are associated with significant unwanted systemic side effects, with direct toxicities on multiple other organ systems at standard dosing, often limiting their broader use.³⁶ In fact, Amiodarone represents ˜30% of the world's antiarrhythmic drug market,³⁷ however nearly ⅓ of all patients cannot tolerate long-term use of the drug due to its extra-cardiac adverse reactions that affect nearly every organ in the body including the thyroid, lungs, liver, eyes, skin, central and peripheral nervous system among others. Direct targeting of antiarrhythmics such as Amiodarone to the CCS has the potential to improve treatment of life-threatening cardiac arrhythmias (e.g. junctional ectopic tachycardia) while at the same time mitigating their serious off-target effects.³⁶ Finally, as the CCS is composed of multiple distinct components, each of which have different functions and are subject to different types of arrhythmias, a true precision approach to antiarrhythmic medication delivery would require the ability to target each subcomponent. As such, here we have uncovered and validated a suite of extracellular markers that can be used for additional targeting efforts (e.g. targeted molecular ablation of the AV node in recalcitrant atrial fibrillation).

Our current work is limited by the need for clinical trials within humans to confirm the degree of resolution and signal penetration in larger, more dense hearts as well as the effect of CCS visualization on patient outcomes. Further, while CNTN2 and NPTN (this work) have been validated in the human CCS, the other extracellular CCS markers described here will need to be verified in humans through the creation of functional human-specific antibodies and/or human single-cell RNA sequencing data of the CCS.

Overall, our study represents, for the first time, a proof-of-principle for antibody-based targeting of molecularly defined cardiac substructures in vivo and lays the groundwork for additional translational opportunities including the targeting of alternative cargos such as contrast agents (MRI/CT), drugs (antiarrhythmics) and other therapeutics (RNA, DNA, small molecules) to the CCS and other cardiac substructures (e.g., atria or ventricles).

### REFERENCES

1. Vedantham V. New Approaches to Biological Pacemakers: Links to Sinoatrial Node Development. Trends Mol Med. 2015; 21:749-761. PMID: 26611337 PMCID: PMC4679473
2. Gross G J, Chiu C C, Hamilton R M, Kirsh J A, Stephenson E A. Natural history of postoperative heart block in congenital heart disease: implications for pacing intervention. Heart Rhythm. 2006; 3:601-604. PMID: 16648069
3. Bonatti V, Agnetti A, Squarcia U. Early and late postoperative complete heart block in pediatric patients submitted to open-heart surgery for congenital heart disease. Pediatr Med Chir. 1998; 20:181-186. PMID: 9744009
4. Romer A J, Tabbutt S, Etheridge S P, Fischbach P, Ghanayem N S, Reddy V M, Sahulee R, Tanel R E, Tweddell J S, Gaies M, Banerjee M, Retzloff L, Zhang W, Patel A R. Atrioventricular block after congenital heart surgery: Analysis from the Pediatric Cardiac Critical Care Consortium. J Thorac Cardiovasc Surg. 2019; 157:1168-1177.e2. PMID: 30917883
5. Karyofillis P, Kostopoulou A, Thomopoulou S, Habibi M, Livanis E, Karavolias G, Voudris V. Conduction abnormalities after transcatheter aortic valve implantation. J Geriatr Cardiol. 2018; 15:105-112. PMID: 29434632 PMCID: PMC5803544
6. Cho Y H, Quintana E, Schaff H V, Nishimura R A, Dearani J A, Abel M D, Ommen S. Residual and recurrent gradients after septal myectomy for hypertrophic cardiomyopathy-mechanisms of obstruction and outcomes of reoperation. J Thorac Cardiovasc Surg. 2014; 148:909-915; discussion 915-916. PMID: 24930617
7. Veselka J, Krejčí J, Tomašov P, Zemánek D. Long-term survival after alcohol septal ablation for hypertrophic obstructive cardiomyopathy: a comparison with general population. Eur Heart J. 2014; 35:2040-2045. PMID: 24464834
8. Vahrmeijer A L, Hutteman M, van der Vorst J R, van de Velde C J H, Frangioni J V. Image-guided cancer surgery using near-infrared fluorescence. Nat Rev Clin Oncol. 2013; 10:507-518. PMID: 23881033 PMCID: PMC3755013
9. Hernot S, van Manen L, Debie P, Mieog J S D, Vahrmeijer A L. Latest developments in molecular tracers for fluorescence image-guided cancer surgery. Lancet Oncol. 2019; 20: e354 e367. PMID: 31267970
10. Rosenthal E L, Warram J M, de Boer E, Chung T K, Korb M L, Brandwein-Gensler M, Strong T V, Schmalbach C E, Morlandt A B, Agarwal G, Hartman Y E, Carroll W R, Richman J S, Clemons L K, Nabell L M, Zinn K R. Safety and Tumor Specificity of Cetuximab-IRDye800 for Surgical Navigation in Head and Neck Cancer. Clin Cancer Res. 2015; 21:3658-3666. PMID: 25904751 PMCID: PMC4909371
11. Zinn K R, Korb M, Samuel S, Warram J M, Dion D, Killingsworth C, Fan J, Schoeb T, Strong T V, Rosenthal E L. IND-directed safety and biodistribution study of intravenously injected cetuximab-IRDye800 in cynomolgus macaques. Mol Imaging Biol. 2015; 17:49-57. PMID: 25080323 PMCID: PMC4318248
12. Pallante B A, Giovannone S, Fang-Yu L, Zhang J, Liu N, Kang G, Dun W, Boyden P A, Fishman G I. Contactin-2 Expression in the Cardiac Purkinje Fiber Network. Circulation: Arrhythmia and Electrophysiology. 2010; 3:186-194.
13. Goodyer W R, Beyersdorf B, Paik D T, Tian L, Li G, Buikema J W, Chirikian O, Choi S, Venkatraman S, Adams E L, Tessier-Lavigne M, Wu J C, Wu S M. Transcriptomic Profiling of the Developing Cardiac Conduction System at Single-Cell Resolution. Circ Res. 2019;PMID: 31284824
14. Waas M, Snarrenberg S T, Littrell J, Jones Lipinski R A, Hansen P A, Corbett J A, Gundry R L. SurfaceGenie: a web-based application for prioritizing cell-type specific marker candidates. Bioinformatics. 2020; PMID: 32053146
15. UniProt Consortium. UniProt: a worldwide hub of protein knowledge. Nucleic Acids Res. 2019; 47: D506-D515. PMID: 30395287 PMCID: PMC6323992
16. Goodyer W R, Gu X, Liu Y, Bottino R, Crabtree G R, Kim S K. Neonatal β cell development in mice and humans is regulated by calcineurin/NFAT. Dev Cell. 2012; 23:21-34. PMID: 22814600 PMCID: PMC3587727
17. Gao R W, Teraphongphom N T, van den Berg N S, Martin B A, Oberhelman N J, Divi V, Kaplan M J, Hong S S, Lu G, Ertsey R, Tummers WSFJ, Gomez A J, Holsinger F C, Kong C S, Colevas A D, Warram J M, Rosenthal E L. Determination of Tumor Margins with Surgical Specimen Mapping Using Near-Infrared Fluorescence. Cancer Res. 2018; 78:5144-5154. PMID: 29967260 PMCID: PMC6125224
18. Pallante B A, Giovannone S, Fang-Yu L, Zhang J, Liu N, Kang G, Dun W, Boyden P A, Fishman G I. Contactin-2 expression in the cardiac Purkinje fiber network. Circ Arrhythm Electrophysiol. 2010; 3:186-194. PMID: 20110552 PMCID: PMC3068837
19. Shekhar A, Lin X, Liu F-Y, Zhang J, Mo H, Bastarache L, Denny J C, Cox N J, Delmar M, Roden D M, Fishman G I, Park D S. Transcription factor ETVI is essential for rapid conduction in the heart. J Clin Invest. 2016; 126:4444-4459. PMID: 27775552 PMCID: PMC5127680
20. Prince A C, Moore L S, Tipirneni K E, Ramesh T, Limdi M A, Bevans S L, Walsh E M, Greene B, Rosenthal E L, Warram J M. Evaluation of optical imaging agents in a fluorescence-guided surgical model of head and neck cancer. Surg Oncol. 2018; 27:225-230. PMID: 29937175 PMCID: PMC6072276
21. Fukamauchi F, Aihara O, Wang Y J, Akasaka K, Takeda Y, Horie M, Kawano H, Sudo K, Asano M, Watanabe K, Iwakura Y. TAG-1-deficient mice have marked elevation of adenosine A1 receptors in the hippocampus. Biochem Biophys Res Commun. 2001; 281:220-226. PMID: 11178983
22. Renier N, Adams E L, Kirst C, Wu Z, Azevedo R, Kohl J, Autry A E, Kadiri L, Umadevi Venkataraju K, Zhou Y, Wang V X, Tang C Y, Olsen O, Dulac C, Osten P, Tessier-Lavigne M. Mapping of Brain Activity by Automated Volume Analysis of Immediate Early Genes. Cell. 2016; 165:1789-1802. PMID: 27238021 PMCID: PMC4912438
23. Mangoni M E, Traboulsie A, Leoni A-L, Couette B, Marger L, Le Quang K, Kupfer E, Cohen-Solal A, Vilar J, Shin H-S, Escande D, Charpentier F, Nargeot J, Lory P. Bradycardia and slowing of the atrioventricular conduction in mice lacking CaV3.1/alpha1G T-type calcium channels. Circ Res. 2006; 98:1422-1430. PMID: 16690884
24. van Eif V W W, Devalla H D, Boink G J J, Christoffels V M. Transcriptional regulation of the cardiac conduction system. Nature Reviews Cardiology. 2018; 15:617-630.
25. Smalla K H, Matthies H, Langnäse K, Shabir S, Böckers™, Wyneken U, Staak S, Krug M, Beesley P W, Gundelfinger E D. The synaptic glycoprotein neuroplastin is involved in long-term potentiation at hippocampal CAI synapses. Proc Natl Acad Sci USA. 2000; 97:4327-4332. PMID: 10759566 PMCID: PMC18241
26. Peretto G, Durante A, Limite L R, Cianflone D. Postoperative arrhythmias after cardiac surgery: incidence, risk factors, and therapeutic management. Cardiol Res Pract. 2014; 2014:615987. PMID: 24511410 PMCID: PMC3912619
27. Sachse F B, Johnson J, Cottle B, Mondal A, Hitchcock R, Kaza A K. Toward detection of conduction tissue during cardiac surgery: Light at the end of the tunnel? Heart Rhythm. 2020; 17:2200-2207. PMID: 32659372 PMCID: PMC7704810
28. Mondal A, Lackey J, Saeed M, Wu F-Y, Johnson J K, Huang C, Sachse F B, Hitchcock R, Kaza A K. An Imaging Protocol to Discriminate Specialized Conduction Tissue During Congenital Heart Surgery. Semin Thorac Cardiovasc Surg. 2019; 31:537-546. PMID: 30738149 PMCID: PMC6684868
29. Buss NAPS, Henderson S J, McFarlane M, Shenton J M, de Haan L. Monoclonal antibody therapeutics: history and future. Curr Opin Pharmacol. 2012; 12:615-622. PMID: 22920732
30. Kurian T, Ambrosi C, Hucker W, Fedorov V V, Efimov I R. Anatomy and electrophysiology of the human A V node. Pacing Clin Electrophysiol. 2010; 33:754 762. PMID: 20180918 PMCID: PMC2889145
31. Sánchez-Quintana D, Yen Ho S. [Anatomy of cardiac nodes and atrioventricular specialized conduction system]. Rev Esp Cardiol. 2003; 56:1085-1092. PMID: 14622540
32. Batteux C, Haidar M A, Bonnet D. 3D-Printed Models for Surgical Planning in Complex Congenital Heart Diseases: A Systematic Review. Front Pediatr [Internet]. 2019 [cited 2021 Jan. 7];7. Available from: https://www.ncbi.nlm.nih.gov/pmc/articles/PMC6378296/PMID: 30805324 PMCID: PMC6378296
33. Baruteau A-E, Abrams D J, Ho S Y, Thambo J-B, McLeod C J, Shah M J. Cardiac Conduction System in Congenitally Corrected Transposition of the Great Arteries and Its Clinical Relevance. J Am Heart Assoc. 2017; 6. PMID: 29269355 PMCID: PMC5779063
34. Hosseini S M, Rozen G, Saleh A, Vaid J, Biton Y, Moazzami K, Heist E K, Mansour M C, Kaadan M I, Vangel M, Ruskin J N. Catheter Ablation for Cardiac Arrhythmias: Utilization and In-Hospital Complications, 2000 to 2013. JACC Clin Electrophysiol. 2017; 3:1240-1248. PMID: 29759619
35. Gasparini M, Kloppe A, Lunati M, Anselme F, Landolina M, Martinez-Ferrer J B, Proclemer A, Morani G, Biffi M, Ricci R, Rordorf R, Mangoni L, Manotta L, Grammatico A, Leyva F, Boriani G. Atrioventricular junction ablation in patients with atrial fibrillation treated with cardiac resynchronization therapy: positive impact on ventricular arrhythmias, implantable cardioverter-defibrillator therapies and hospitalizations. Eur J Heart Fail. 2018; 20:1472-1481. PMID: 29251799
36. Jalife J, Stevenson W G, Zipes D P, editors. Cardiac electrophysiology—from cell to bedside. Seventh edition. [7th ed. Philadelphia, P A: Elsevier; 2018.
37. Allen LaPointe N M, Dai D, Thomas L, Piccini J P, Peterson E D, Al-Khatib S M. Antiarrhythmic drug use in patients <65 years with atrial fibrillation and without structural heart disease. Am J Cardiol. 2015; 115:316-322. PMID: 25491240 PMCID: PMC4293335

### SEQUENCE LISTING

Sequence Number (ID): 1
Length: 108
Molecule Type: AA
Features Location/Qualifiers:
   REGION, 1 . . . 108
      note, Synthetic: a light chain variable region V_{L} of an antibody having a specific binding activity to human contactin 2
   source, 1 . . . 108
      mol_type, protein
      organism, synthetic construct
Residues:
EIVLTQSPGT LSLSPGERAT LSCRASQSVS SSYLAWYQQK PGQAPRLLIY GASSRATGIP 60
DRFSGSGSGT DFTLTISRLE PEDFAVYYCQ QYGSSPWTFG QGTKVEIK 108
Sequence Number (ID): 2
Length: 325
Molecule Type: DNA
Features Location/Qualifiers:
   misc_feature, 1 . . . 325
      note, Synthetic: a cDNA sequence encoding the light chain variable region V_{L} with SEQ ID NO: 1
   source, 1 . . . 325
      mol_type, other DNA
      organism, synthetic construct
Residues:
gaaattgtgt tgacacagtc tccaggcacc ctgtctttgt ctccagggga aagagccacc 60
ctctcctgca gggccagtca gagtgttagc agcagctact tagcctggta ccagcagaaa 120
cctggccagg ctcccaggct cctcatctat ggtgcatcca gcagggccac tggcatccca 180
gacaggttca gtggcagtgg gtctgggaca gacttcactc tcaccatcag cagactggag 240
cctgaagatt ttgcagtgta ttactgtcag cagtatggta gctcaccttg gacgttcggc 300
caagggacca aggtggaaat caaac 325
Sequence Number (ID): 3
Length: 117
Molecule Type: AA
Features Location/Qualifiers:
   REGION, 1 . . . 117
      note, Synthetic: a heavy chain variable region V_{H} of an antibody having a specific binding activity to human contactin 2
   source, 1 . . . 117
      mol_type, protein
      organism, synthetic construct
Residues:
QVHLVESGGG LLQPGGSLRL SCAASGFTFS SYAMSWVRQA PGKGLEWVSG IVGNGGSTYY 60
ADSVKGRFTI SRDNSKNTLY LOMNSLRAED TAVYYCAKLE WGALGYWGQG TLVTVSS 117
Sequence Number (ID): 4
Length: 351
Molecule Type: DNA
Features Location/Qualifiers:
   misc_feature, 1 . . . 351
      note, Synthetic: a cDNA sequence encoding the heavy chain variable region V_{H} with SEQ ID NO: 3
   source, 1 . . . 351
      mol_type, other DNA
      organism, synthetic construct
Residues:
caggtgcacc tggtggagtc tgggggaggc ttgctacagc ctggggggtc cctgagactc 60
tcctgtgcag cctctggatt cacttttagc agctatgcca tgagctgggt ccgccaggct 120
ccagggaagg ggctggagtg ggtctcaggt attgttggta atggtggtag cacatactac 180
gcagactccg tgaaaggccg gttcaccatc tccagagaca attccaagaa cacactgtat 240
ctgcaaatga acagcctgag agccgaggac accgccgtat attactgtgc gaaattggag 300
tggggggcct tggggtactg gggccaggga accctggtca ccgtctcaag c 351
Sequence Number (ID): 5
Length: 112
Molecule Type: AA
Features Location/Qualifiers:
   REGION, 1 . . . 112
      note, Synthetic: a light chain variable region V_{L} of an antibody having a specific binding activity to human contactin 2
   source, 1 . . . 112
      mol_type, protein
      organism, synthetic construct
Residues:
DVVMTQSPLS LPVTLGQPAS ISCRSSQSLV YSDGNTYLYW FQQRPGQSPR RLIYQVSNRD 60
SGVPDRFSGS GSGTDFTLKI SRVEAEDVGV YYCMQALETP ITFGQGTRLE IK 112
Sequence Number (ID): 6
Length: 337
Molecule Type: DNA
Features Location/Qualifiers:
   misc_feature, 1 . . . 337
      note, Synthetic: a cDNA sequence encoding the light chain variable region V_{L} with SEQ ID NO: 5
   source, 1 . . . 337
      mol_type, other DNA
      organism, synthetic construct
Residues:
gatgttgtga tgactcagtc tccactctcc ctgcccgtca cccttggaca gccggcctcc 60
atctcctgca ggtctagtca aagcctcgta tacagtgatg gaaacaccta cttgtattgg 120
tttcagcaga ggccaggcca atctccaagg cgcctaattt atcaggtttc taaccgggac 180
tctggggtcc cagacagatt cagcggcagt gggtcaggca ctgatttcac actgaaaatc 240
agcagggtgg aggctgagga tgttggggtt tattactgca tgcaagctct agaaactccg 300
atcaccttcg gccaagggac acgactggag attaaac 337
Sequence Number (ID): 7
Length: 120
Molecule Type: AA
Features Location/Qualifiers:
   REGION, 1 . . . 120
      note, Synthetic: a heavy chain variable region V_{H} of an antibody having a specific binding activity to human contactin 2
   source, 1 . . . 120
      mol_type, protein
      organism, synthetic construct
Residues:
EVOLVETGGG LIQPGGSLRL SCAASGFTVS SNYMSWVRQA PGKGLEWVSV IYSGGSTYYA 60
DSVKGRFTIS RDNSKNTLYL QMNSLRAEDT AVYYCARDRI AAAGTLPVYW GQGTLVTVSS 120
Sequence Number (ID): 8
Length: 360
Molecule Type: DNA
Features Location/Qualifiers:
   misc_feature, 1 . . . 360
      note, Synthetic: a cDNA sequence encoding the heavy chain variable region V_{H} with SEQ ID NO: 7
   source, 1 . . . 360
      mol_type, other DNA
      organism, synthetic construct
Residues:
gaggtgcagc tggtggagac cggaggaggc ttgatccagc ctggggggtc cctgagactc 60
tcctgtgcag cctctgggtt caccgtcagt agcaactaca tgagctgggt ccgccaggct 120
ccagggaagg ggctggagtg ggtctcagtt atttatagcg gtggtagcac atactacgca 180
gactccgtga agggccgatt caccatctcc agagacaatt ccaagaacac gctgtatctt 240
caaatgaaca gcctgagagc cgaggacacg gccgtgtatt actgtgcgag agaccgtata 300
gcagcagctg gtacccttcc cgtctactgg ggccagggca ccctggtcac cgtctcaagc 360
Sequence Number (ID): 9
Length: 1040
Molecule Type: AA
Features Location/Qualifiers:
   REGION, 1 . . . 1040
      note, misc_feature—amino acid sequence of human contactin 2 (CNTN2)
   source, 1 . . . 1040
      mol_type, protein
      organism, Homo sapiens
Residues:
MGTATRRKPH LLLVAAVALV SSSAWSSALG SQTTFGPVFE DQPLSVLFPE ESTEEQVLLA 60
CRARASPPAT YRWKMNGTEM KLEPGSRHQL VGGNLVIMNP TKAQDAGVYQ CLASNPVGTV 120
VSREAILRFG FLOEFSKEER DPVKAHEGWG VMLPCNPPAH YPGLSYRWLL NEFPNFIPTD 180
GRHFVSQTTG NLYIARTNAS DLGNYSCLAT SHMDFSTKSV FSKFAQLNLA AEDTRLFAPS 240
IKARFPAETY ALVGQQVTLE CFAFGNPVPR IKWRKVDGSL SPQWTTAEPT LQIPSVSFED 300
EGTYECEAEN SKGRDTVQGR IIVQAQPEWL KVISDTEADI GSNLRWGCAA AGKPRPTVRW 360
LRNGEPLASQ NRVEVLAGDL RFSKLSLEDS GMYQCVAENK HGTIYASAEL AVQALAPDFR 420
LNPVRRLIPA ARGGEILIPC QPRAAPKAVV LWSKGTEILV NSSRVTVTPD GTLIIRNISR 480
SDEGKYTCFA ENFMGKANST GILSVRDATK ITLAPSSADI NLGDNLTLQC HASHDPTMDL 540
TFTWTLDDFP IDFDKPGGHY RRTNVKETIG DLTILNAQLR HGGKYTCMAQ TVVDSASKEA 600
TVLVRGPPGP PGGVVVRDIG DTTIQLSWSR GFDNHSPIAK YTLQARTPPA GKWKQVRTNP 660
ANIEGNAETA QVLGLTPWMD YEFRVIASNI LGTGEPSGPS SKIRTREAAP SVAPSGLSGG 720
GGAPGELIVN WTPMSREYQN GDGFGYLLSF RRQGSTHWQT ARVPGADAQY FVYSNESVRP 780
YTPFEVKIRS YNRRGDGPES LTALVYSAEE EPRVAPTKVW AKGVSSSEMN VTWEPVQQDM 840
NGILLGYEIR YWKAGDKEAA ADRVRTAGLD TSARVSGLHP NTKYHVTVRA YNRAGTGPAS 900
PSANATTMKP PPRRPPGNIS WTFSSSSLSI KWDPVVPFRN ESAVTGYKML YQNDLHLTPT 960
LHLTGKNWIE IPVPEDIGHA LVQIRTTGPG GDGIPAEVHI VRNGGTSMMV ENMAVRPAPH 1020
PGTVISHSVA MLILIGSLEL 1040
Sequence Number (ID): 10
Length: 12
Molecule Type: AA
Features Location/Qualifiers:
   REGION, 1 . . . 12
      note, Synthetic: CDR1 fragment of a light chain variable region V_{L} of an antibody having a specific binding activity to human contactin 2
   source, 1 . . . 12
      mol_type, protein
      organism, synthetic construct
Residues:
RASQSVSSSY LA 12
Sequence Number (ID): 11
Length: 8
Molecule Type: AA
Features Location/Qualifiers:
   REGION, 1 . . . 8
      note, Synthetic: CDR2 fragment of a light chain variable region V_{L} of an antibody having a specific binding activity to human contactin 2
   source, 1 . . . 8
      mol_type, protein
      organism, synthetic construct
Residues:
YGASSRAT 8
Sequence Number (ID): 12
Length: 9
Molecule Type: AA
Features Location/Qualifiers:
   REGION, 1 . . . 9
      note, Synthetic: CDR3 fragment of a light chain variable region V_{L} of an antibody having a specific binding activity to human contactin 2
   source, 1 . . . 9
      mol_type, protein
      organism, synthetic construct
Residues:
QQYGSSPWT 9
Sequence Number (ID): 13
Length: 13
Molecule Type: AA
Features Location/Qualifiers:
   REGION, 1 . . . 13
      note, Synthetic: CDR1 fragment of a heavy chain variable region V_{H} of an antibody having a specific binding activity to human contactin 2
   source, 1 . . . 13
      mol_type, protein
      organism, synthetic construct
Residues: LSCAASGFTF SSY 13
Sequence Number (ID): 14
Length: 10
Molecule Type: AA
Features Location/Qualifiers:
   REGION, 1 . . . 10
      note, Synthetic: CDR2 fragment of a heavy chain variable region V_{H} of an antibody having a specific binding activity to human contactin 2
   source, 1 . . . 10
      mol_type, protein
      organism, synthetic construct
Residues:
WVSGIVGNGG 10
Sequence Number (ID): 15
Length: 16
Molecule Type: AA
Features Location/Qualifiers:
   REGION, 1 . . . 16
      note, Synthetic: CDR1 fragment of a light chain variable region V_{L} of an antibody having a specific binding activity to human contactin 2
   source, 1 . . . 16
      mol_type, protein
      organism, synthetic construct
Residues:
RSSQSLVYSD GNTYLY 16
Sequence Number (ID): 16
Length: 8
Molecule Type: AA
Features Location/Qualifiers:
   REGION, 1 . . . 8
      note, Synthetic: CDR2 fragment of a light chain variable region V_{L} of an antibody having a specific binding activity to human contactin 2
   source, 1 . . . 8
      mol_type, protein
      organism, synthetic construct
Residues:
YQVSNRDS 8
Sequence Number (ID): 17
Length: 9
Molecule Type: AA
Features Location/Qualifiers:
   REGION, 1 . . . 9
      note, Synthetic: CDR3 fragment of a light chain variable region V_{L} of an antibody having a specific binding activity to human contactin 2
   source, 1 . . . 9
      mol_type, protein
      organism, synthetic construct
Residues:
MQALETPIT 9
Sequence Number (ID): 18
Length: 13
Molecule Type: AA
Features Location/Qualifiers:
   REGION, 1 . . . 13
      note, Synthetic: CDR1 fragment of a heavy chain variable region V_{H} of an antibody having a specific binding activity to human contactin 2
   source, 1 . . . 13
      mol_type, protein
      organism, synthetic construct
Residues:
AASGFTVSSN YMS 13
Sequence Number (ID): 19
Length: 9
Molecule Type: AA
Features Location/Qualifiers:
   REGION, 1 . . . 9
      note, Synthetic: CDR2 fragment of a heavy chain variable region V_{H} of an antibody having a specific binding activity to human contactin 2
   source, 1 . . . 9
      mol_type, protein
      organism, synthetic construct
Residues:
VIYSGGSTY 9
Sequence Number (ID): 20
Length: 982
Molecule Type: AA
Features Location/Qualifiers:
   REGION, 1 . . . 982
      note, Synthetic: Recombinant human CNTN2 fragment
   source, 1 . . . 982
      mol_type, protein
      organism, synthetic construct
Residues:
SQTTFGPVFE DQPLSVLFPE ESTEEQVLLA CRARASPPAT YRWKMNGTEM KLEPGSRHQL 60
VGGNLVIMNP TKAQDAGVYQ CLASNPVGTV VSREAILRFG FLQEFSKEER DPVKAHEGWG 120
VMLPCNPPAH YPGLSYRWLL NEFPNFIPTD GRHFVSQTTG NLYIARTNAS DLGNYSCLAT 180
SHMDFSTKSV FSKFAQLNLA AEDTRLFAPS IKARFPAETY ALVGQQVTLE CFAFGNPVPR 240
IKWRKVDGSL SPQWTTAEPT LQIPSVSFED EGTYECEAEN SKGRDTVQGR IIVQAQPEWL 300
KVISDTEADI GSNLRWGCAA AGKPRPTVRW LRNGEPLASQ NRVEVLAGDL RFSKLSLEDS 360
GMYQCVAENK HGTIYASAEL AVQALAPDFR LNPVRRLIPA ARGGEILIPC QPRAAPKAVV 420
LWSKGTEILV NSSRVTVTPD GTLIIRNISR SDEGKYTCFA ENFMGKANST GILSVRDATK 480
ITLAPSSADI NLGDNLTLQC HASHDPTMDL TFTWTLDDFP IDFDKPGGHY RRTNVKETIG 540
DLTILNAQLR HGGKYTCMAQ TVVDSASKEA TVLVRGPPGP PGGVVVRDIG DTTIQLSWSR 600
GFDNHSPIAK YTLQARTPPA GKWKQVRTNP ANIEGNAETA QVLGLTPWMD YEFRVIASNI 660
LGTGEPSGPS SKIRTREAAP SVAPSGLSGG GGAPGELIVN WTPMSREYQN GDGFGYLLSF 720
RRQGSTHWQT ARVPGADAQY FVYSNESVRP YTPFEVKIRS YNRRGDGPES LTALVYSAEE 780
EPRVAPTKVW AKGVSSSEMN VTWEPVQQDM NGILLGYEIR YWKAGDKEAA ADRVRTAGLD 840
TSARVSGLHP NTKYHVTVRA YNRAGTGPAS PSANATTMKP PPRRPPGNIS WTFSSSSLSI 900
KWDPVVPFRN ESAVTGYKML YQNDLHLTPT LHLTGKNWIE IPVPEDIGHA LVQIRTTGPG 960
GDGIPAEVHI VRNGGTSMMV EN 982
Sequence Number (ID): 21
Length: 344
Molecule Type: AA
Features Location/Qualifiers:
   REGION, 1 . . . 344
      note, misc_feature—amino acid sequence of human Neurotrimin (NTM)
   source, 1 . . . 344
      mol_type, protein
      organism, Homo sapiens
Residues:
MGVCGYLFLP WKCLVVVSLR LLFLVPTGVP VRSGDATFPK AMDNVTVRQG ESATLRCTID 60
NRVTRVAWLN RSTILYAGND KWCLDPRVVL LSNTQTQYSI EIQNVDVYDE GPYTCSVQTD 120
NHPKTSRVHL IVQVSPKIVE ISSDISINEG NNISLTCIAT GRPEPTVTWR HISPKAVGFV 180
SEDEYLEIQG ITREQSGDYE CSASNDVAAP VVRRVKVTVN YPPYISEAKG TGVPVGQKGT 240
LQCEASAVPS AEFQWYKDDK RLIEGKKGVK VENRPFLSKL IFFNVSEHDY GNYTCVASNK 300
LGHTNASIML FGPGAVSEVS NGTSRRAGCV WLLPLLVLHL LLKF 344
Sequence Number (ID): 22
Length: 398
Molecule Type: AA
Features Location/Qualifiers:
   REGION, 1 . . . 398
      note, misc_feature—amino acid sequence of human Neuroplastin (NPTN)
   source, 1 . . . 398
      mol_type, protein
      organism, Homo sapiens
Residues:
MSGSSLPSAL ALSLLLVSGS LLPGPGAAQN AGFVKSPMSE TKLTGDAFEL YCDVVGSPTP 60
EIQWWYAEVN RAESFROLWD GARKRRVTVN TAYGSNGVSV LRITRLTLED SGTYECRASN 120
DPKRNDLRQN PSITWIRAQA TISVLOKPRI VTSEEVIIRD SPVLPVTLQC NLTSSSHTLT 180
YSYWTKNGVE LSATRKNASN MEYRINKPRA EDSGEYHCVY HFVSAPKANA TIEVKAAPDI 240
TGHKRSENKN EGQDATMYCK SVGYPHPDWI WRKKENGMPM DIVNTSGRFF IINKENYTEL 300
NIVNLQITED PGEYECNATN AIGSASVVTV LRVRSHLAPL WPFLGILAEI IILVVIIVVY 360
EKRKRPDEVP DDDEPAGPMK TNSTNNHKDK NLRQRNTN 398

1. An antibody or an antigen-binding fragment thereof, having a specific binding activity to human contactin 2 (CNTN2) protein with the amino acid sequence of SEQ ID NO: 9, the antibody or the antigen-binding fragment thereof comprising:
a). at least a first polypeptide comprising a light chain variable region (V_{L}); and
b). at least a second polypeptide comprising a heavy chain variable region (V_{H}),
wherein the first polypeptide is linked to the second polypeptide,
wherein the light chain variable region (V_{L}) of the first polypeptide comprises at least one from the following four amino acid sequences:
1. the amino acid sequence with SEQ ID NO: 1 or a variant thereof with at least 70% amino acid sequence identity to SEQ ID NO: 1;
2. the amino acid sequence with SEQ ID NO: 5 or a variant thereof with at least 70% amino acid sequence identity to SEQ ID NO: 5;
3. an amino acid sequence comprising Complementarity-Determining Region 1 (CDR1) with SEQ ID NO: 10 or a variant thereof with at least 90% amino acid sequence identity to SEQ ID NO: 10, Complementarity-Determining Region 2 (CDR2) with SEQ ID NO: 11 or a variant thereof with at least 90% amino acid sequence identity to SEQ ID NO: 11, and Complementarity-Determining Region 3 (CDR3) with SEQ ID NO: 13 or a variant thereof with at least 90% amino acid sequence identity to SEQ ID NO: 13; or
4. an amino acid sequence comprising CDR1 with SEQ ID NO: 15 or a variant thereof with at least 90% sequence identity to SEQ ID NO: 15, CDR2 with SEQ ID NO: 16 or a variant thereof with at least 90% sequence identity to SEQ ID NO: 16, and CDR3 with SEQ ID NO: 17 or a variant thereof with at least 90% sequence identity to SEQ ID NO: 17; and
wherein the heavy chain variable region (V_{H}) of the second polypeptide comprises at least one from the following four amino acid sequences:
1. an amino acid sequence with SEQ ID NO: 3 or a variant thereof with at least 70% amino acid sequence identity to SEQ ID NO: 3;
2. an amino acid sequence with SEQ ID NO: 7 or a variant thereof with at least 70% amino acid sequence identity to SEQ ID NO: 7;
3. an amino acid sequence comprising CDR1 with SEQ ID NO: 13 or a variant thereof having at least 90% sequence identity to SEQ ID NO: 13, and CDR2 with SEQ ID NO: 14 or a variant thereof with at least 90% identity to SEQ ID NO: 14; or
4. an amino acid sequence comprising CDR1 with SEQ ID NO: 18 or a variant thereof with at least 90% sequence identity to SEQ ID NO: 18, and CDR2 with SEQ ID NO: 19 or a variant thereof with at least 90% sequence identity to SEQ ID NO: 19.

2. The antibody or the antigen-binding fragment thereof of claim 1, wherein the first polypeptide is linked to the second polypeptide by a disulfide bond and/or wherein the antibody or the antigen-binding fragment further comprises a third polypeptide comprising the light chain variable region (V_{L}), the third polypeptide being linked to a fourth polypeptide, the fourth polypeptide comprising the heavy variable region (VII) and wherein the second polypeptide and the fourth polypeptide are linked.

3. The antibody or the antigen-binding fragment thereof of claim 1, wherein the first polypeptide further comprises at least a portion of a light chain constant region (C_{L}) and wherein the second polypeptide further comprises a least a portion of a heavy chain constant region (CIII).

4. The antibody or the antigen-binding fragment thereof of claim 1, wherein the antibody or the antigen-binding fragment thereof comprises:
two polypeptides a). and
two polypeptides b)., and
wherein the two polypeptides b). are linked.

5. The antibody or the antigen-binding fragment thereof of claim 1, wherein the antibody or the antigen-binding fragment thereof is a human monoclonal antibody.

6. The antibody or the antigen-binding fragment thereof of claim 1, wherein the antibody or an antigen-binding fragment thereof is a human monoclonal fragment antigen-binding region (Fab) composed of the first polypeptide linked to the second polypeptide, wherein the first polypeptide further comprises at least a portion of a light chain constant region (C_{L}) and wherein the second polypeptide further comprises a least a portion of a heavy chain constant region (C_{H1}).

7. The antibody or the antigen-binding fragment thereof of claim 1,
wherein the first polypeptide comprises the amino acid sequence with SEQ ID NO: 1 or 5, or a variant amino acid sequence with at least 90% or at least 95% amino acid sequence identity to SEQ ID NO: 1 or 5; and
wherein the second polypeptide comprises the amino acid sequence with SEQ ID NO: 3 or 7, or a variant amino acid sequence with at least 90% or at least 95% amino acid sequence identity to SEQ ID NO: 3 or 7.

8. A reagent useful for therapeutic, imaging and/or diagnostic applications, the reagent comprising the antibody or the antigen binding fragment of claim 1 conjugated with one or more of a detection agent and/or a therapeutic drug.

9. The regent of claim 8, wherein the antibody or the antigen binding fragment is conjugated with a near-infrared dye (NIR), a superparamagnetic iron oxide nanoparticle (SPION), a gold nanoparticle, saporin, an antiarrhythmic drug, a ribonucleic acid (RNA), deoxyribonucleic acid (DNA) or CAS9 enzyme-single guide complex.

10. The regent of claim 8, wherein the antibody or the antigen binding fragment is conjugated directly or indirectly by being displayed on the surface of a carrier.

11. The regent of claim 10, wherein the carrier comprises a liposome.

12. The regent of claim 8, wherein the antibody or the antigen binding fragment is conjugated directly or indirectly to amiodarone, procainamide or near-infrared dye.

13. A composition comprising the antibody or the antigen-binding fragment thereof of claim 1, and one or more excipients.

14. The composition of claim 13, wherein
the composition is formulated for intravenous or intracoronary injection.

15. The composition of claim 13, wherein
the composition comprises from 0.1 wt % to 99.9 wt % of the antibody or the antigen-binding fragment thereof of claim 1 or the regent of claim 8, and from 0.1 wt % to 99.9 wt % of the one or more excipients.

16. A method of treating a subject, the method comprising administering to the subject the composition of claim 13.

17. The method of claim 16, wherein the composition is administered orally, topically, locally or systemically and/or wherein the composition is administered in an amount from about 0.05 mg to about 100 mg of the detection agent and/or the therapeutic drug per one kilogram of the subject's body weight.

18. A recombinant nucleic acid comprising the nucleic acid with SEQ ID NO. 2, SEQ ID NO. 4, SEQ ID NO. 6 and/or SEQ ID NO. 8, or a nucleic acid variant with at least 70% nucleic acid sequence identity with SEQ ID NO. 2, SEQ ID NO. 4, SEQ ID NO. 6 and/or SEQ ID NO. 8.

19. A recombinant cell comprising the recombinant nucleic acid of claim 18.

20. A method for visualizing the cardiac conduction system (CCS) in a subject, the method comprising:
a) administering to the subject the composition of claim 8 and/or administering to the subject a composition comprising one or more antibodies with a specific binding activity to human neurotrimin (Ntm) or human neuroplastin (Nptn); and
b) visualizing the CCS in the subject in real time by contacting the subject with ultrasound, computed tomography, visible, UV and/or infrared light.


SEQUENCE LISTING (22 sequences)

SEQ ID NO: 1 - protein, 108 aa, synthetic construct
  REGION 1..108: note = Synthetic: a light chain variable region VL of an antibody having a specific binding activity to human contactin 2
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 2 - DNA, 325 bp, synthetic construct
  misc_feature 1..325: note = Synthetic: a cDNA sequence encoding the light chain variable region VL with SEQ ID NO: 1
  source 1..325: mol_type = other DNA
gaaattgtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaac

SEQ ID NO: 3 - protein, 117 aa, synthetic construct
  REGION 1..117: note = Synthetic: a heavy chain variable region VH of an antibody having a specific binding activity to human contactin 2
QVHLVESGGGLLQPGGSLRLSCAASGFTFSSYAMSWVRQAPGKGLEWVSGIVGNGGSTYYADSVKGRFTISRDNSKNTLYLQMNSLRAEDTAVYYCAKLEWGALGYWGQGTLVTVSS

SEQ ID NO: 4 - DNA, 351 bp, synthetic construct
  misc_feature 1..351: note = Synthetic: a cDNA sequence encoding the heavy chain variable region VH with SEQ ID NO: 3
  source 1..351: mol_type = other DNA
caggtgcacctggtggagtctgggggaggcttgctacagcctggggggtccctgagactctcctgtgcagcctctggattcacttttagcagctatgccatgagctgggtccgccaggctccagggaaggggctggagtgggtctcaggtattgttggtaatggtggtagcacatactacgcagactccgtgaaaggccggttcaccatctccagagacaattccaagaacacactgtatctgcaaatgaacagcctgagagccgaggacaccgccgtatattactgtgcgaaattggagtggggggccttggggtactggggccagggaaccctggtcaccgtctcaagc

SEQ ID NO: 5 - protein, 112 aa, synthetic construct
  REGION 1..112: note = Synthetic: a light chain variable region VL of an antibody having a specific binding activity to human contactin 2
DVVMTQSPLSLPVTLGQPASISCRSSQSLVYSDGNTYLYWFQQRPGQSPRRLIYQVSNRDSGVPDRFSGSGSGTDFTLKISRVEAEDVGVYYCMQALETPITFGQGTRLEIK

SEQ ID NO: 6 - DNA, 337 bp, synthetic construct
  misc_feature 1..337: note = Synthetic: a cDNA sequence encoding the light chain variable region VL with SEQ ID NO: 5
  source 1..337: mol_type = other DNA
gatgttgtgatgactcagtctccactctccctgcccgtcacccttggacagccggcctccatctcctgcaggtctagtcaaagcctcgtatacagtgatggaaacacctacttgtattggtttcagcagaggccaggccaatctccaaggcgcctaatttatcaggtttctaaccgggactctggggtcccagacagattcagcggcagtgggtcaggcactgatttcacactgaaaatcagcagggtggaggctgaggatgttggggtttattactgcatgcaagctctagaaactccgatcaccttcggccaagggacacgactggagattaaac

SEQ ID NO: 7 - protein, 120 aa, synthetic construct
  REGION 1..120: note = Synthetic: a heavy chain variable region VH of an antibody having a specific binding activity to human contactin 2
EVQLVETGGGLIQPGGSLRLSCAASGFTVSSNYMSWVRQAPGKGLEWVSVIYSGGSTYYADSVKGRFTISRDNSKNTLYLQMNSLRAEDTAVYYCARDRIAAAGTLPVYWGQGTLVTVSS

SEQ ID NO: 8 - DNA, 360 bp, synthetic construct
  misc_feature 1..360: note = Synthetic: a cDNA sequence encoding the heavy chain variable region VH with SEQ ID NO: 7
  source 1..360: mol_type = other DNA
gaggtgcagctggtggagaccggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagaccgtatagcagcagctggtacccttcccgtctactggggccagggcaccctggtcaccgtctcaagc

SEQ ID NO: 9 - protein, 1040 aa, Homo sapiens
  REGION 1..1040: note = misc_feature - amino acid sequence of human contactin 2 (CNTN2)
MGTATRRKPHLLLVAAVALVSSSAWSSALGSQTTFGPVFEDQPLSVLFPEESTEEQVLLACRARASPPATYRWKMNGTEMKLEPGSRHQLVGGNLVIMNPTKAQDAGVYQCLASNPVGTVVSREAILRFGFLQEFSKEERDPVKAHEGWGVMLPCNPPAHYPGLSYRWLLNEFPNFIPTDGRHFVSQTTGNLYIARTNASDLGNYSCLATSHMDFSTKSVFSKFAQLNLAAEDTRLFAPSIKARFPAETYALVGQQVTLECFAFGNPVPRIKWRKVDGSLSPQWTTAEPTLQIPSVSFEDEGTYECEAENSKGRDTVQGRIIVQAQPEWLKVISDTEADIGSNLRWGCAAAGKPRPTVRWLRNGEPLASQNRVEVLAGDLRFSKLSLEDSGMYQCVAENKHGTIYASAELAVQALAPDFRLNPVRRLIPAARGGEILIPCQPRAAPKAVVLWSKGTEILVNSSRVTVTPDGTLIIRNISRSDEGKYTCFAENFMGKANSTGILSVRDATKITLAPSSADINLGDNLTLQCHASHDPTMDLTFTWTLDDFPIDFDKPGGHYRRTNVKETIGDLTILNAQLRHGGKYTCMAQTVVDSASKEATVLVRGPPGPPGGVVVRDIGDTTIQLSWSRGFDNHSPIAKYTLQARTPPAGKWKQVRTNPANIEGNAETAQVLGLTPWMDYEFRVIASNILGTGEPSGPSSKIRTREAAPSVAPSGLSGGGGAPGELIVNWTPMSREYQNGDGFGYLLSFRRQGSTHWQTARVPGADAQYFVYSNESVRPYTPFEVKIRSYNRRGDGPESLTALVYSAEEEPRVAPTKVWAKGVSSSEMNVTWEPVQQDMNGILLGYEIRYWKAGDKEAAADRVRTAGLDTSARVSGLHPNTKYHVTVRAYNRAGTGPASPSANATTMKPPPRRPPGNISWTFSSSSLSIKWDPVVPFRNESAVTGYKMLYQNDLHLTPTLHLTGKNWIEIPVPEDIGHALVQIRTTGPGGDGIPAEVHIVRNGGTSMMVENMAVRPAPHPGTVISHSVAMLILIGSLEL

SEQ ID NO: 10 - protein, 12 aa, synthetic construct
  REGION 1..12: note = Synthetic: CDR1 fragment of a light chain variable region VL of an antibody having a specific binding activity to human contactin 2
RASQSVSSSYLA

SEQ ID NO: 11 - protein, 8 aa, synthetic construct
  REGION 1..8: note = Synthetic: CDR2 fragment of a light chain variable region VL of an antibody having a specific binding activity to human contactin 2
YGASSRAT

SEQ ID NO: 12 - protein, 9 aa, synthetic construct
  REGION 1..9: note = Synthetic: CDR3 fragment of a light chain variable region VL of an antibody having a specific binding activity to human contactin 2
QQYGSSPWT

SEQ ID NO: 13 - protein, 13 aa, synthetic construct
  REGION 1..13: note = Synthetic: CDR1 fragment of a heavy chain variable region VH of an antibody having a specific binding activity to human contactin 2
LSCAASGFTFSSY

SEQ ID NO: 14 - protein, 10 aa, synthetic construct
  REGION 1..10: note = Synthetic: CDR2 fragment of a heavy chain variable region VH of an antibody having a specific binding activity to human contactin 2
WVSGIVGNGG

SEQ ID NO: 15 - protein, 16 aa, synthetic construct
  REGION 1..16: note = Synthetic: CDR1 fragment of a light chain variable region VL of an antibody having a specific binding activity to human contactin 2
RSSQSLVYSDGNTYLY

SEQ ID NO: 16 - protein, 8 aa, synthetic construct
  REGION 1..8: note = Synthetic: CDR2 fragment of a light chain variable region VL of an antibody having a specific binding activity to human contactin 2
YQVSNRDS

SEQ ID NO: 17 - protein, 9 aa, synthetic construct
  REGION 1..9: note = Synthetic: CDR3 fragment of a light chain variable region VL of an antibody having a specific binding activity to human contactin 2
MQALETPIT

SEQ ID NO: 18 - protein, 13 aa, synthetic construct
  REGION 1..13: note = Synthetic: CDR1 fragment of a heavy chain variable region VH of an antibody having a specific binding activity to human contactin 2
AASGFTVSSNYMS

SEQ ID NO: 19 - protein, 9 aa, synthetic construct
  REGION 1..9: note = Synthetic: CDR2 fragment of a heavy chain variable region VH of an antibody having a specific binding activity to human contactin 2
VIYSGGSTY

SEQ ID NO: 20 - protein, 982 aa, synthetic construct
  REGION 1..982: note = Synthetic: Recombinant human CNTN2 fragment
SQTTFGPVFEDQPLSVLFPEESTEEQVLLACRARASPPATYRWKMNGTEMKLEPGSRHQLVGGNLVIMNPTKAQDAGVYQCLASNPVGTVVSREAILRFGFLQEFSKEERDPVKAHEGWGVMLPCNPPAHYPGLSYRWLLNEFPNFIPTDGRHFVSQTTGNLYIARTNASDLGNYSCLATSHMDFSTKSVFSKFAQLNLAAEDTRLFAPSIKARFPAETYALVGQQVTLECFAFGNPVPRIKWRKVDGSLSPQWTTAEPTLQIPSVSFEDEGTYECEAENSKGRDTVQGRIIVQAQPEWLKVISDTEADIGSNLRWGCAAAGKPRPTVRWLRNGEPLASQNRVEVLAGDLRFSKLSLEDSGMYQCVAENKHGTIYASAELAVQALAPDFRLNPVRRLIPAARGGEILIPCQPRAAPKAVVLWSKGTEILVNSSRVTVTPDGTLIIRNISRSDEGKYTCFAENFMGKANSTGILSVRDATKITLAPSSADINLGDNLTLQCHASHDPTMDLTFTWTLDDFPIDFDKPGGHYRRTNVKETIGDLTILNAQLRHGGKYTCMAQTVVDSASKEATVLVRGPPGPPGGVVVRDIGDTTIQLSWSRGFDNHSPIAKYTLQARTPPAGKWKQVRTNPANIEGNAETAQVLGLTPWMDYEFRVIASNILGTGEPSGPSSKIRTREAAPSVAPSGLSGGGGAPGELIVNWTPMSREYQNGDGFGYLLSFRRQGSTHWQTARVPGADAQYFVYSNESVRPYTPFEVKIRSYNRRGDGPESLTALVYSAEEEPRVAPTKVWAKGVSSSEMNVTWEPVQQDMNGILLGYEIRYWKAGDKEAAADRVRTAGLDTSARVSGLHPNTKYHVTVRAYNRAGTGPASPSANATTMKPPPRRPPGNISWTFSSSSLSIKWDPVVPFRNESAVTGYKMLYQNDLHLTPTLHLTGKNWIEIPVPEDIGHALVQIRTTGPGGDGIPAEVHIVRNGGTSMMVEN

SEQ ID NO: 21 - protein, 344 aa, Homo sapiens
  REGION 1..344: note = misc_feature - amino acid sequence of human Neurotrimin (NTM)
MGVCGYLFLPWKCLVVVSLRLLFLVPTGVPVRSGDATFPKAMDNVTVRQGESATLRCTIDNRVTRVAWLNRSTILYAGNDKWCLDPRVVLLSNTQTQYSIEIQNVDVYDEGPYTCSVQTDNHPKTSRVHLIVQVSPKIVEISSDISINEGNNISLTCIATGRPEPTVTWRHISPKAVGFVSEDEYLEIQGITREQSGDYECSASNDVAAPVVRRVKVTVNYPPYISEAKGTGVPVGQKGTLQCEASAVPSAEFQWYKDDKRLIEGKKGVKVENRPFLSKLIFFNVSEHDYGNYTCVASNKLGHTNASIMLFGPGAVSEVSNGTSRRAGCVWLLPLLVLHLLLKF

SEQ ID NO: 22 - protein, 398 aa, Homo sapiens
  REGION 1..398: note = misc_feature - amino acid sequence of human Neuroplastin (NPTN)
MSGSSLPSALALSLLLVSGSLLPGPGAAQNAGFVKSPMSETKLTGDAFELYCDVVGSPTPEIQWWYAEVNRAESFRQLWDGARKRRVTVNTAYGSNGVSVLRITRLTLEDSGTYECRASNDPKRNDLRQNPSITWIRAQATISVLQKPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHLAPLWPFLGILAEIIILVVIIVVYEKRKRPDEVPDDDEPAGPMKTNSTNNHKDKNLRQRNTN